(19) United States
(12) Patent Application Publication
(10) Pub. No.: US 2023/0047934 A1
(43) Pub. Date: Feb. 16, 2023

(54) HUMAN TISSUE DERIVED COMPOSITIONS AND USES THEREOF

(71) Applicant: Osiris Therapeutics, Inc., Columbia, MD (US)

(72) Inventors: Steven Michael SINCLAIR, Ellicott City, MD (US); Alla DANILKOVITCH, Columbia, MD (US); Malathi SATHYAMOORTHY, Ellicott City, MD (US); Jin-Qiang KUANG, Glenelg, MD (US); Sandeep DHALL, Elkridge, MD (US); Yishan LIU, Doylestown, PA (US); Anthony John MELCHIORRI, Ellicott City, MD (US); Matthew Robert MOORMAN, Chestertown, MD (US); Mena Schiano LO MORIELLO, Silver Spring, MD (US); Anne Allgood LERCH, Annapolis, MD (US)

(73) Assignee: Osiris Therapeutics, Inc., Columbia, MD (US)

(21) Appl. No.: 17/788,939

(22) PCT Filed: Mar. 12, 2021
(86) PCT No.: PCT/US2021/022059

§ 371 Date: Jun. 24, 2022

Related U.S. Application Data
(60) Provisional application No. 62/988,490, filed on Mar. 12, 2020.

(51) Int. Cl. A61K 35/50; A61K 35/33; A61K 35/51; A61K 9/19; A61K 9/00

(52) U.S. Cl. CPC A61K 35/50; A61K 35/33; A61K 35/51; A61K 9/19; A61K 9/0019

(57) ABSTRACT

Disclosed are compositions comprising a minced chorionic matrix, a homogenized amniotic matrix, and a homogenized umbilical cord (UC) matrix, wherein the minced chorionic matrix comprises viable cells, wherein the composition does not comprise trophoblasts. Disclosed are compositions comprising isolated, viable chorionic cells, a homogenized amniotic matrix, and a homogenized UC matrix, wherein the composition does not comprise trophoblasts. Disclosed are compositions comprising a minced chorionic matrix and a homogenized UC matrix, wherein the minced chorionic matrix comprises viable cells, wherein the composition does not comprise trophoblasts or an amniotic matrix. Disclosed are compositions comprising isolated, viable chorionic cells and a homogenized

UC matrix, wherein the composition does not comprise trophoblasts or an amniotic matrix. Disclosed are methods of treating a tissue injury or chronic pain comprising administering any of the disclosed compositions to an area of a subject comprising a tissue injury.

### CROSS REFERENCE TO RELATED APPLICATIONS

This application claims priority to U.S. Provisional Application No. 62/988,490 filed Mar. 12, 2020. The contents of the referenced application are incorporated into the present application by reference.

### BACKGROUND OF THE INVENTION

The use of placental tissues for burns and other types of wounds originated more than 100 years ago. Placental tissues contain components that are present in skin and other tissues and required for wound healing or tissue regeneration such as extracellular matrix, growth factors, and cells, including MSCs that are responsible for orchestrating the healing process in different tissue types. The effectiveness of placental tissues such as amniotic and chorionic membranes for burns, ocular wounds, orthopedic, and sports medicine surgical applications has been recorded in a number of published reports; however, the use of fresh placental tissues for a variety of indications is limited due to challenges of short shelf-life.

What is needed in the art is a therapeutic product that provides the benefits of placental tissues yet can be applied in flowable forms that is compatible with delivery via injection or minimally invasive techniques such as arthroscopy, endoscopy, or laprascopy. Furthermore, a therapeutic product that contains matrix proteins, growth factors, and viable placental cells that will dynamically respond to the injury and aid in tissue regeneration is desired. Flowable forms of such therapeutics could be used to produce a solid matrix, which can be prepared into most shapes and sizes. The methods and materials described herein can provide a solution to such needs.

### SUMMARY OF THE INVENTION

Disclosed are compositions comprising a minced chorionic matrix, a homogenized amniotic matrix, and a homogenized umbilical cord (UC) matrix, wherein the minced chorionic matrix comprises viable cells. In some aspects, the composition does not comprise trophoblasts.

Disclosed are compositions comprising isolated, viable chorionic cells, a homogenized amniotic matrix, and a homogenized UC matrix. In some aspects, the composition does not comprise trophoblasts.

Disclosed are compositions comprising a minced chorionic matrix and a homogenized UC matrix, wherein the minced chorionic matrix comprises viable cells. In some aspects, the composition does not comprise trophoblasts or an amniotic matrix.

Disclosed are compositions comprising isolated, viable chorionic cells and a homogenized UC matrix. In some aspects, the composition does not comprise trophoblasts or an amniotic matrix.

Disclosed are compositions comprising isolated, viable chorionic cells, a homogenized UC matrix, and a homogenized amniotic matrix. In some aspects, the composition does not comprise trophoblasts.

Disclosed are pharmaceutical compositions comprising any one of the disclosed compositions and a pharmaceutically acceptable carrier.

Disclosed are methods of making the disclosed compositions comprising preparing a minced chorionic matrix; preparing a homogenized UC matrix; and combining the minced chorionic matrix and the homogenized UC matrix

Disclosed are methods of making the disclosed compositions comprising preparing isolated chorionic cells; preparing a homogenized UC matrix; and combining the isolated chorionic cells and the homogenized UC matrix.

Disclosed are methods of treating a tissue injury or chronic pain comprising administering one or more of the disclosed compositions to an area of a subject comprising a tissue injury.

Also disclosed are Embodiments 1 to 81 of the present invention. Embodiment 1 is a composition comprising a minced chorionic matrix, a homogenized amniotic matrix, and a homogenized umbilical cord (UC) matrix, wherein the minced chorionic matrix comprises viable cells, wherein the composition does not comprise trophoblasts. Embodiment 2 is the composition of Embodiment 1, wherein the homogenized amniotic matrix is devitalized. Embodiment 3 is the composition of Embodiments 1 to 2, wherein the homogenized umbilical cord (UC) matrix is devitalized. Embodiment 4 is the composition of Embodiments 1 to 3, wherein the chorionic matrix, homogenized amniotic matrix and homogenized UC matrix are from the same donor. Embodiment 5 is the composition of Embodiments 1 to 4, wherein at least two of the chorionic matrix, homogenized amniotic matrix and homogenized UC matrix are from the same donor.

Embodiment 6 is a composition comprising isolated, viable chorionic cells, a homogenized amniotic matrix, and a homogenized UC matrix, wherein the composition does not comprise trophoblasts. Embodiment 7 is the composition of Embodiment 6, wherein the homogenized amniotic matrix is devitalized. Embodiment 8 is the composition of Embodiments 6 to 7, wherein the homogenized UC matrix is devitalized. Embodiment 9 is the composition of Embodiments 6 to 8, wherein the homogenized amniotic matrix and homogenized UC matrix are derived from the same donor. Embodiment 10 is the composition of Embodiments 6 to 9, wherein the isolated chorionic cells and homogenized amniotic matrix are from the same donor. Embodiment 11 is the composition of Embodiments 6 to 10, wherein the isolated chorionic cells and homogenized UC matrix are from the same donor. Embodiment 12 is the composition of Embodiments 6 to 11, wherein the isolated chorionic cells, homogenized amniotic matrix, and homogenized UC matrix are from the same donor. Embodiment 13 is the composition of Embodiments 6 to 12, wherein the isolated chorionic cells comprise greater than or equal to 100,000 viable cells/ml. Embodiment 14 is the composition of Embodiments 6 to 13, wherein the isolated chorionic cells have not been culturally expanded. Embodiment 15 is the composition of Embodiments 6 to 14, wherein the composition further comprises a chorionic matrix. Embodiment 16 is the composition of Embodiments 6 to 15, wherein the composition further comprises a non-homogenized chorionic matrix. Embodiment 17 is the composition of Embodiments 6 to 16, wherein the composition further comprises a minced chorionic matrix. Embodiment 18 is the composition of Embodiments 6 to 17, wherein the composition further comprises a minced chorionic matrix comprising native, viable cells. Embodiment 19 is the composition of Embodiments 6 to 18, wherein the composition further comprises a minced chorionic matrix comprising viable cells that have not been culturally expanded.

Embodiment 20 is a composition comprising a minced chorionic matrix and a homogenized UC matrix, wherein the minced chorionic matrix comprises viable cells, wherein the composition does not comprise trophoblasts or an amniotic matrix. Embodiment 21 is the composition of Embodiment 20, wherein the homogenized UC matrix is devitalized. Embodiment 22 is the composition of Embodiments 20 to 21, wherein the minced chorionic matrix and homogenized UC matrix are from the same donor. Embodiment 23 is the composition of Embodiments 20 to 22, wherein the minced chorionic matrix is non-homogenized.

Embodiment 24 is a composition comprising isolated, viable chorionic cells and a homogenized UC matrix, wherein the composition does not comprise trophoblasts or an amniotic matrix. Embodiment 25 is the composition of Embodiment 24, wherein the homogenized UC matrix is devitalized. Embodiment 26 is the composition of Embodiments 24 to 25, wherein the isolated chorionic cells and homogenized UC matrix are from the same donor. Embodiment 27 is the composition of Embodiments 24 to 26, wherein the isolated chorionic cells comprise greater than or equal to 100,000 viable cells/ml. Embodiment 28 is the composition of Embodiments 24 to 27, wherein the isolated chorionic cells have not been culturally expanded. Embodiment 29 is the composition of Embodiments 24 to 28, wherein the composition further comprises a chorionic matrix. Embodiment 30 is the composition of Embodiments 24 to 29, wherein the composition further comprises a non-homogenized chorionic matrix. Embodiment 31 is the composition of Embodiments 24 to 30, wherein the composition further comprises a minced chorionic matrix.

Embodiment 32 is a composition comprising isolated, viable chorionic cells, a homogenized UC matrix, and a homogenized amniotic matrix wherein the composition does not comprise trophoblasts. Embodiment 33 is the composition of Embodiment 32, wherein the homogenized UC matrix is devitalized. Embodiment 34 is the composition of Embodiments 32 to 33, wherein the isolated chorionic cells and homogenized UC matrix are from the same donor. Embodiment 35 is the composition of Embodiments 32 to 34, wherein the isolated chorionic cells comprise greater than or equal to 100,000 viable cells/ml. Embodiment 36 is the composition of Embodiments 32 to 35, wherein the isolated chorionic cells have not been culturally expanded. Embodiment 37 is the composition of Embodiments 32 to 36, wherein the composition further comprises a chorionic matrix. Embodiment 38 is the composition of Embodiments 32 to 37, wherein the composition further comprises a non-homogenized chorionic matrix. Embodiment 39 is the composition of Embodiments 32 to 38, wherein the composition further comprises a minced chorionic matrix.

Embodiment 40 is the composition of Embodiments 1 to 5, 20 to 23, 29 to 31, and 37 to 39 wherein the chorionic matrix comprises viable cells. Embodiment 41 is the composition of Embodiment 40, wherein the chorionic matrix comprises native, viable cells. Embodiment 42 is the composition of Embodiments 40 to 41, wherein the chorionic matrix comprises viable cells that have not been culturally expanded.

Embodiment 43 is the composition of Embodiments 1 to 5, 15 to 23, and 29 to 31, and 37 to 42 wherein the chorionic matrix comprises greater than or equal to 100,000 viable cells/ml.

Embodiment 44 is the composition of Embodiments 1 to 43, wherein the composition comprises viable chorionic stem cells, amniotic stem cells, fibroblasts, epithelial cells, or a combination thereof. Embodiment 45 is the composition of Embodiments 1 to 44, wherein the composition is formulated as a cream, gel, oil, ointment, or lotion. Embodiment 46 is the composition of Embodiments 1 to 45, wherein the composition further comprises a viscous modifier. Embodiment 47 is the composition of Embodiments 1 to 46, wherein composition further comprises a viscous modifier comprising hyaluronic acid, methylcellulose, carboxymethylcellulose, xanthum gum, pluronics, thermally responsive polymers and proteins, fibronectins, laminins, collagens, chitosan, or chondroitin sulfate. Embodiment 48 is the composition of Embodiments 1 to 47, wherein the composition further comprises viable, isolated amniotic cells. Embodiment 49 is the composition of Embodiments 1 to 48, wherein the composition further comprises a scaffold. Embodiment 50 is the composition of Embodiments 1 to 49, wherein the composition further comprises a natural or synthetic scaffold. Embodiment 51 is the composition of Embodiments 1 to 50, wherein the composition further comprises a scaffold derived from skin, hyaline cartilage, meniscus, intervertebral discF, or bone. Embodiment 52 is the composition of Embodiments 1 to 51, wherein the composition further comprises a natural or synthetic polymer scaffold. Embodiment 53 is the composition of Embodiments 1 to 52, wherein the homogenized UC matrix comprises de-veined UC tissue. Embodiment 54 is the composition of Embodiments 1 to 53, wherein the composition is cryopreserved. Embodiment 55 is the composition of Embodiments 1 to 54, wherein the composition comprises a cryopreservation solution. Embodiment 56 is the composition of Embodiments 1 to 55, wherein the composition is lyophilized. Embodiment 57 is the composition of Embodiments 1 to 56, wherein the composition further comprises a pharmaceutically acceptable excipient.

Embodiment 58 is a pharmaceutical composition comprising the composition of Embodiments 1 to 56 and a pharmaceutically acceptable carrier.

Embodiment 59 is a method of making the composition of Embodiments 1-5, 20-22, and 35-57, the method comprising: a) preparing a minced chorionic matrix; b) preparing a homogenized UC matrix; and c) combining the minced chorionic matrix and the homogenized UC matrix. Embodiment 60 is the method of Embodiment 59, wherein the method further comprises preparing a homogenized amniotic matrix and combining the homogenized amniotic matrix with the minced chorionic matrix and the homogenized UC matrix. Embodiment 61 is a method of making the composition of Embodiments 6 to 19 and 23 to 33 comprising: a) preparing isolated chorionic cells; b) preparing a homogenized UC matrix; and c) combining the isolated chorionic cells and the homogenized UC matrix. Embodiment 62 is the method of Embodiment 61, wherein preparing isolated chorionic cells comprises isolating chorionic cells from chorionic tissue. Embodiment 63 is the method of Embodiments 61 to 62, wherein the method further comprises the step of preparing a homogenized amniotic matrix and combining with the isolated chorionic cells and the homogenized UC matrix. Embodiment 64 is the method of Embodiments 61 to 63, wherein the method further comprises preparing a non-homogenized chorionic matrix. Embodiment 65 is the method of Embodiments 61 to 64, wherein the method further comprises preparing a non-homogenized chorionic matrix by mincing chorionic tissue. Embodiment 66 is the method of Embodiments 61 to 65, wherein the method further comprises preparing a non-homogenized chorionic matrix by removing a trophoblast layer. Embodiment 67 is the method of Embodiments 61 to 66, wherein the isolated chorionic cells and UC tissue are derived from the same donor. Embodiment 68 is the method of Embodiments 59 to 67, wherein the method further comprises adding a viscous modifier. Embodiment 69 is the method of Embodiments 59 to 68, wherein the homogenized UC matrix comprises de-veined UC tissue. Embodiment 70 is the method of Embodiments 59 to 69, wherein the method further comprises adding a scaffold. Embodiment 71 is the method of Embodiments 59 to 70, wherein the method further comprises adding a natural or synthetic scaffold. Embodiment 72 is the method of Embodiments 59 to 71, wherein the method further comprises adding a scaffold derived from a meniscus, a disc, or bone. Embodiment 73 is the method of Embodiments 59 to 72, wherein the method further comprises adding a natural or synthetic polymer scaffold. Embodiment 74 is the method of Embodiments 59 to 73, wherein the method further comprises preparing a homogenized amniotic matrix and combining the homogenized amniotic matrix with the minced chorionic matrix and the homogenized UC matrix, and prior to preparing a homogenized amniotic matrix, isolating epithelial cells from the amniotic matrix. Embodiment 75 is the method of Embodiments 59 to 74, wherein the method further comprises preparing a homogenized amniotic matrix and combining the homogenized amniotic matrix with the minced chorionic matrix and the homogenized UC matrix; prior to preparing a homogenized amniotic matrix, isolating epithelial cells from the amniotic matrix; and combining the isolated amniotic epithelial cells to the combined isolated chorionic cells, the homogenized amniotic matrix, and the homogenized UC matrix. Embodiment 76 is the method of Embodiments 59 to 75, wherein the method further comprises lyophilizing the combined isolated chorionic cells and UC matrix.

Embodiment 77 is a method of treating a tissue injury or chronic pain comprising administering the composition of Embodiments 1 to 58 to an area of a subject comprising a tissue injury. Embodiment 78 is the method of Embodiment 77, wherein the tissue injury is osteoarthritis, cartilage repair, meniscus repair, intervertebral disc repair, plantar fasciitis, carpal tunnel, tendonitis, synovitis, ruptured or torn Achilles tendon, critical limb ischemia, ulcers, pyoderma gangrenosum, epidermolysis bullosa, surgical adhesions, plastic surgery or other wounds. Embodiment 79 is the method of Embodiments 77 to 78, wherein the composition is administered by injecting the composition to the area of a subject comprising a tissue injury or local region of pain or inflammation. Embodiment 80 is the method of Embodiments 77 to 79, wherein the composition is administered by applying the composition topically to an area of a subject comprising the tissue injury or pain or inflammation. Embodiment 81 is the method of Embodiments 77 to 80, wherein the composition is administered by implanting the composition to the area of a subject comprising a tissue injury.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the percent TNF-alpha inhibition of a composition comprising a minced chorionic matrix, a homogenized amniotic matrix, and a homogenized UC matrix.

FIG. 2 shows the levels of TSG-6 in minced chorion.

FIG. 3 shows the levels of TSG-6 in isolated chorion cells cultured overnight.

FIG. 4 shows the percent TNF-alpha inhibition of a composition comprising a minced chorionic matrix and a homogenized UC matrix.

FIG. 5 shows the percent TNF-alpha inhibition of a composition comprising isolated chorion cells and a homogenized UC matrix.

FIG. 6 shows an example of the staining of fresh (no HSA), 24 hr HSA incubation, and 48 hr HSA incubation of minced chorion tissue showing the presence of live cells, using the Live/Dead staining method, and n=2 representative images.

FIGS. 7A, 7B, and 7C are a schematic of the different compositions with their intended uses. A) Injectable—examples of indications are: knee osteoarthritis, plantar fasciitis, achilles tendon repair, critical limb ischemia, plastic procedures, diabetic foot ulcers (DFUs), venous leg ulcers (VLUs), pressure ulcers, pyoderma gangrenosum, epidermolysis bullosa, other wounds, plastic procedures. B) Topical- examples of indications are DFUs, VLUs, pressure ulcers, pyoderma gangrenosum, epidermolysis bullosa, other wounds, plastic procedures; C) Surgical—examples of indications are meniscus repair, disc repair, plastic reconstructions, cartilage repair, surgical adhesion barriers for laprascopic or open procedures in gynecology, urology, bariatrics, or similar fields, and bone repair.

FIG. 8 shows bioengineered platform building blocks.

FIG. 9 shows a composition/product for chronic wounds. The composition/product is a lyophilized flowable formulation of chorionic matrix containing viable tissue native cells mixed with umbilical cord and amniotic matrix. The composition/product was stored at room temperature and was reconstituted with saline solution prior to application.

FIG. 10 is a diagram of an example of how to process full-term placenta with UC.

FIG. 11 depicts the histological appearance of the individual placental tissues and the final compositions containing viable non-homogenized chorionic components and homogenized placental matrix.

FIG. 12 shows the high cell viability of the non-homogenized chorionic components of compositions before and after preservation by lyophilization.

FIG. 13 demonstrates the lack of an immunogenic response to the compositions due to the selective depletion or devitalization of immunogenic cell types.

FIG. 14 summarizes the FACS analysis of cells isolated from the non-homogenized viable chorionic component of the compositions.

### DETAILED DESCRIPTION OF THE INVENTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a chorionic matrix” includes a plurality of such matrices, reference to “the umbilical cord matrix” is a reference to one or more umbilical cord matrix and equivalents thereof known to those skilled in the art, and so forth.

The term “homogenized” means to make substantially similar in size and composition For example, if a portion of a homogenized tissue matrix was removed after homogenization, the overall morphology and macromolecular make up in the portion removed and in the remaining homogenized tissue matrix would be substantially similar in size and composition. For example, a “homogenized amniotic matrix” and “homogenized umbilical cord matrix” can mean that the amnion or umbilical cord samples have been processed to a point that the entire sample is comprised of particles smaller than 1 mm in diameter (hydrodynamic radius of 0.5 mm), and preferably small enough to pass through an 18-gauge needle (inner diameter of 0.838 mm) without requiring significant syringe plunger pressure, as well as soluble factors homogeneously distributed through the sample. In some aspects, a homogenized tissue can be a tissue that has been previously homogenized wherein the homogenized tissue can have particle sizes within the ranges of 10 nm to 1mm, or preferably 100 μm to 500 μm, 1 μm to 100 μm, 100 nm to 1 μm, or 10 nm to 100 nm. Generally, methods used for homogenization apply more power (energy over time) to the compositions than non-homogenization methods, such as mincing.

The term “devitalized tissue matrix” refers to tissue matrix that is substantially or completely devoid of viable or live cells. Where a tissue matrix is not substantially or completely devitalized, the tissue matrix retains a population of viable or live cells. Devitalized tissue matrix is achieved by killing viable or live cells present in the tissue matrix.

“Mincing” means to cut to make similar in size and composition; however mincing generally results in less uniformity and larger particles than homogenization or homogenized tissue (e.g. range of particle sizes is broader than the range of particle sizes of a homogenized tissue). “Minced tissue” refers to tissue that has been minced and is generally less uniform and has larger average particle sizes than homogenized tissue.

“Digesting” refers to the process of using enzymes to disaggregate primary tissue (i.e., chorionic tissue) to achieve performance of single cell isolation while retaining maximum cell viability. When the enzymes come in contact with the tissue, the matrix can be loosened, and cells can be released from the tissue. Tissue exposure to the enzymes can be minimized to avoid adverse effects on cell viability. Tissues can be partially or completely digested. “Digested tissue” refers to primary tissue that has been subjected to enzymatic disaggregation to obtain isolated cells.

The phrase “isolated cells” refers to cells removed or isolated from tissue. For example, “isolated chorionic cells” are cells removed or isolated from chorionic tissue. Isolated cells can refer to any population of cells, for example, epithelial cells and/or stromal fibroblasts or stromal MSCs, with at least 30%, 40%, 50%, 60%, 70%, 80%, 90%, or 95% cell viability. Thus, “isolated cells” can also be referred to as “isolated, viable cells” (e.g. isolated, viable chorionic cells).

“Chorionic matrix” refers to tissue derived from the chorion. Chorionic matrix can include homogenized, non-homogenized, or minced chorionic matrix. Chorionic matrix and chorionic tissue can be used interchangeably.

“UC matrix” refers to tissue derived from the umbilical cord. UC matrix can include homogenized or non-homogenized UC matrix. UC matrix and UC tissue can be used interchangeably.

“Amniotic matrix” refers to tissue derived from the amnion. Amniotic matrix can include homogenized or non-homogenized amniotic matrix. Amniotic matrix and amniotic tissue can be used interchangeably.

“Optional” or “optionally” means that the subsequently described event, circumstance, or material may or may not occur or be present, and that the description includes instances where the event, circumstance, or material occurs or is present and instances where it does not occur or is not present.

“Native cells” means cells that are native, resident, or endogenous to the tissue sample or composition, i.e. cells that are not exogenously added (e.g, seeded) to a tissue sample or composition as described herein.

“Substantially free” means present in only a negligible amount or not present at all. For example, when a cell is abundant less than about 20% or less than about 10% or less than about 1% of the amount in an unprocessed sample.

“Therapeutic cells” as used herein means viable cells native to a given tissue or composition as described herein that have retained their native biological functions to dynamically respond to a local microenvironment, for example an injury site or wound.

Examples of therapeutic cells include, but are not limited to, fibroblasts, epithelial cells, MSCs, and other tissue-specific cell types, such as osteoblasts or osteoclasts for bone, or CD34+ follicular cells of the skin epidermis, or chondrocytes of hyaline cartilage, or fibrochondrocytes of meniscus, or annulus fibrosus or nucleus pulposus cells of the intervertebral disc, or supportive cell types surrounding peripheral nerve.

“Therapeutic factors” means tissue-derived factors that promote wound healing or tissue regeneration. For example, placenta- or chorionic membrane-derived factors that promote wound healing or tissue regeneration. Examples include, but are not limited to IGFBP1, adiponectin, α2-macroglobulin, and bFGF. Other examples include, but are not limited to MMP-9 and TIMP1. Other therapeutic factors include, but are not limited to, TGF-beta 1, beta 2, or beta 3, HGF, VEGF, IGF-1, and BMPs.

“Stromal cells” refers to a mixed population of cells present (optionally in native proportions) composed of mesenchymal stem cells and fibroblasts natively found within the stromal layer of a given tissue type.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

The compositions of the present disclosure and methods for their use can “comprise,” “consist essentially of,” or “consist of” any of the ingredients or steps disclosed throughout the specification. With respect to the phrase “consist essentially of,” a basic and novel property of the compositions of the present disclosure are their ability to treat tissue injuries or chronic pain by administration of any of the disclosed compositions to the area of a subject having a tissue injury.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### B. Compositions

1. Compositions Comprising a Chorionic Matrix, an Amniotic Matrix and an UC Matrix, Wherein the Minced Chorionic Matrix Comprises Viable Cells

Disclosed are compositions comprising a non-homogenized chorionic matrix, a homogenized amniotic matrix and a homogenized umbilical cord (UC) matrix, wherein the non-homogenized chorionic matrix comprises viable cells.

In some aspects, the non-homogenized chorionic matrix can be minced chorionic matrix. Thus, disclosed are compositions comprising a minced chorionic matrix, a homogenized amniotic matrix and a homogenized UC matrix, wherein the minced chorionic matrix comprises viable cells.

Disclosed are compositions comprising a minced chorionic matrix, a homogenized amniotic matrix and a homogenized UC matrix, wherein the minced chorionic matrix comprises viable cells and further comprising viable, isolated amniotic cells. Thus, disclosed are compositions comprising a minced chorionic matrix, isolated, viable amniotic epithelial cells, a homogenized amniotic matrix and a homogenized UC matrix, wherein the minced chorionic matrix comprises viable cells. In some aspects, the isolated, viable amniotic epithelial cells are from the same amniotic tissue as the homogenized amniotic matrix. In some aspects, the isolated, viable amniotic epithelial cells are from a different amniotic tissue as the homogenized amniotic matrix.

In some aspects, disclosed are compositions comprising a minced chorionic matrix, a non-homogenized amniotic matrix and a homogenized UC matrix, wherein the minced chorionic matrix comprises viable cells. Also disclosed are compositions comprising a minced chorionic matrix, a non-homogenized amniotic matrix and a homogenized UC matrix, wherein the minced chorionic matrix comprises viable cells and wherein the composition further comprises viable, isolated amniotic cells. In some aspects, non-homogenized amniotic matrix can be minced.

In some aspects, disclosed are compositions comprising a minced chorionic matrix, a homogenized amniotic matrix and a non-homogenized UC matrix, wherein the minced chorionic matrix comprises viable cells. Also disclosed are compositions comprising a minced chorionic matrix, a homogenized amniotic matrix and a non-homogenized UC matrix, wherein the non-homogenized chorionic matrix comprises viable cells and wherein the composition further comprises viable, isolated amniotic cells. In some aspects, non-homogenized UC matrix can be minced.

In some aspects, disclosed are compositions comprising a minced chorionic matrix, a non-homogenized amniotic matrix and a non-homogenized UC matrix, wherein the minced chorionic matrix comprises viable cells. Also disclosed are compositions comprising a minced chorionic matrix, a non-homogenized amniotic matrix and a non-homogenized UC matrix, wherein the minced chorionic matrix comprises viable cells and wherein the composition further comprises viable, isolated amniotic cells. In some aspects, non-homogenized amniotic matrix and/or the non-homogenized UC matrix can be minced.

Thus, in some aspects, the disclosed compositions can comprise a variety of components that include a minced chorionic matrix, a non-homogenized or homogenized amniotic matrix, a non-homogenized or homogenized UC matrix, wherein the minced chorionic matrix comprises viable cells. In some aspects, the disclosed compositions can comprise a variety of components that include a minced chorionic matrix, a non-homogenized or homogenized amniotic matrix, a non-homogenized or homogenized UC matrix, wherein the minced chorionic matrix comprises viable cells and wherein the composition further comprises viable, isolated amniotic cells.

Chorionic matrix is gently prepared in order to preserve cell viability of the chorionic cells. Thus, the chorionic matrix is a non-homogenized chorionic matrix. In some aspects, non-homogenized chorionic matrix can be minced.

In some aspects, minced chorionic matrix can comprise native, viable cells. In some aspects, the native, viable cells have not been culturally expanded. In some aspects, the native, viable cells have never been removed from the chorionic matrix. In some aspects, minced chorionic matrix can comprise viable cells that have not been culturally expanded. In some aspects, minced chorionic matrix is not substantially devitalized. Minced chorionic matrix can comprise some dead cells. In some aspects, minced chorionic matrix can comprise greater than 50%, 60%, 70%, 80%, 90%, 95% viable cells. In some aspects, the minced chorionic matrix can comprise greater than or equal to 100,000 viable cells/ml. In some aspects, the ratio of viable chorionic cells to all other nonviable cells in the composition can be 5:1, 2:1, 1:1, 1:2, 1:5, 1:10, 1:20, 1:30, 1:40, 1:50, 1:60, 1:70, 1:80, 1:90 or 1:100.

In some aspects, amniotic cells are isolated from the amniotic tissue prior to homogenizing or mincing and then the isolated amniotic cells are added back to the disclosed compositions. Thus, when isolated amniotic cells are added back into the composition, the ratio of viable chorionic cells to all other nonviable cells is higher because there are less nonviable cells because the amniotic cells can be still viable after being isolated and combined back into the composition.

In some aspects, the homogenized amniotic matrix and/or the homogenized UC matrix are not decellularized. In some aspects, the homogenized amniotic matrix and/or the homogenized UC matrix are devitalized. Thus, the homogenized amniotic matrix and/or the homogenized UC matrix can comprise non-viable cells.

In some aspects, at least two of the chorionic matrix, homogenized amniotic matrix and homogenized UC matrix are from the same donor. In some aspects, the homogenized amniotic matrix and homogenized UC matrix can be derived from the same donor. In some aspects, the minced chorionic matrix and homogenized amniotic matrix can be derived from the same donor. In some aspects, the minced chorionic matrix and homogenized UC matrix can be derived from the same donor. In some aspects, the minced chorionic matrix and homogenized amniotic matrix and homogenized UC matrix can be derived from the same donor. In some aspects, each of the minced chorionic matrix and homogenized amniotic matrix and homogenized UC matrix can be derived from different donors. In some aspects, at least one of the minced chorionic matrix and homogenized amniotic matrix and homogenized UC matrix is from a different donor than the other two matrices.

In some aspects, the disclosed compositions can comprise viable chorionic stem cells, fibroblasts, epithelial cells or a combination thereof.

In some aspects, the homogenized UC matrix comprises de-veined UC tissue.

2. Compositions Comprising Isolated, Viable Chorionic Cells, an Amniotic Matrix, and a UC Matrix, Wherein the Composition Does Not Comprise Trophoblasts

Disclosed are compositions comprising isolated, viable chorionic cells, a homogenized amniotic matrix, and a homogenized UC matrix, wherein the composition does not comprise trophoblasts.

In some aspects, disclosed are compositions comprising isolated, viable chorionic cells, a non-homogenized amniotic matrix and a homogenized UC matrix. Also disclosed are compositions comprising isolated, viable chorionic cells, a non-homogenized amniotic matrix and a homogenized UC matrix, wherein the composition further comprises viable, isolated amniotic cells. In some aspects, non-homogenized amniotic matrix can be minced.

In some aspects, disclosed are compositions comprising isolated, viable chorionic cells, a homogenized amniotic matrix and a non-homogenized UC matrix. Also disclosed are compositions comprising isolated, viable chorionic cells, a homogenized amniotic matrix and a non-homogenized UC matrix, wherein the composition further comprises viable, isolated amniotic cells. In some aspects, non-homogenized UC matrix can be minced.

In some aspects, disclosed are compositions comprising isolated, viable chorionic cells, a non-homogenized amniotic matrix and a non-homogenized UC matrix. Also disclosed are compositions comprising isolated, viable chorionic cells, a non-homogenized amniotic matrix and a non-homogenized UC matrix, wherein the composition further comprises viable, isolated amniotic cells. In some aspects, non-homogenized amniotic matrix and/or the non-homogenized UC matrix can be minced.

Thus, in some aspects, the disclosed compositions can comprise a variety of components that include isolated, viable chorionic cells, a non-homogenized or homogenized amniotic matrix, a non-homogenized or homogenized UC matrix. In some aspects, the disclosed compositions can comprise a variety of components that include isolated, viable chorionic cells, a non-homogenized or homogenized amniotic matrix, a non-homogenized or homogenized UC matrix, wherein the composition further comprises viable, isolated amniotic cells.

In some aspects, the amniotic matrix and/or the UC matrix, regardless of whether one or both are homogenized or non-homogenized, are not decellularized. In some aspects, the amniotic matrix and/or the UC matrix, regardless of whether one or both are homogenized or non-homogenized, are decellularized.In some aspects, the amniotic matrix and/or the UC matrix, regardless of whether one or both are homogenized or non-homogenized, are devitalized. Thus, the amniotic matrix and/or the UC matrix, regardless of whether one or both are homogenized or non-homogenized, can comprise non-viable cells.

In some aspects, at least two of the isolated chorionic cells, homogenized amniotic matrix and homogenized UC matrix are from the same donor. In some aspects, the homogenized amniotic matrix and homogenized UC matrix can be derived from the same donor. In some aspects, the isolated chorionic cells and homogenized amniotic matrix can be derived from the same donor. In some aspects, the isolated chorionic cells and homogenized UC matrix can be derived from the same donor. In some aspects, the isolated chorionic cells and homogenized amniotic matrix and homogenized UC matrix can be derived from the same donor. In some aspects, each of the isolated chorionic cells and homogenized amniotic matrix and homogenized UC matrix can be derived from different donors. In some aspects, at least one of the isolated chorionic cells and homogenized amniotic matrix and homogenized UC matrix is from a different donor than the other two.

In some aspects, the isolated chorionic cells comprise greater than or equal to 100,000 viable cells/ml. In some aspects, the isolated chorionic cells comprise a range of 25,000 to 1,000,000 viable cells. In some aspects, the isolated chorionic cells comprise a range of 50,000 to 750,000 viable cells. In some aspects, the isolated chorionic cells comprise a range of 25,000 to 500,000 viable cells. In some aspects, the isolated chorionic cells comprise a range of 100,000 to 500,000.

In some aspects, the isolated chorionic cells have not been culturally expanded. In some aspects the isolated chorionic cells can be washed and resuspended but they are not cultured. For example, the isolated chorionic cells can be washed and/or resuspended in cell culture media as long as they are not cultured (e.g. placed under conditions that allow for cell growth). In some aspects, the isolated chorionic cells can be cultured. For example, the isolated chorionic cells can be passaged one, two, three, four or five times. Thus, in some aspects, the isolated chorionic cells are from P1, P2, P3, P4, or P5. The early passaged cells can retain their inherent characteristics and therefore are very similar to non-cultured cells.

Disclosed are compositions comprising isolated, viable chorionic cells, a homogenized amniotic matrix, and a homogenized UC matrix, wherein the composition does not comprise trophoblasts, and further comprising a chorionic matrix. In some aspects, the chorionic matrix is non-homogenized. In some aspects, the chorionic matrix is minced. In some aspects, the minced chorionic matrix comprises native, viable cells. In some spects, the native, viable cells have been isolated from the chorionic matrix prior to mincing and then added back in to the chorionic matrix. In some aspects, the native, viable cells have never been isolated or removed from the chorionic matrix. In some aspects, the minced chorionic matrix comprises viable cells that have not been culturally expanded.

In some aspects, the disclosed compositions can comprise viable chorionic stem cells, fibroblasts, epithelial cells or a combination thereof.

In some aspects, the homogenized UC matrix comprises de-veined UC tissue.

3. Compositions Comprising a Minced Chorionic Matrix and a Homogenized UC Matrix, Wherein the Minced Chorionic Matrix Comprises Viable Cells, Wherein the Composition Does Not Comprise Trophoblasts or an Amniotic Matrix

Disclosed are compositions comprising a minced chorionic matrix and a homogenized UC matrix, wherein the minced chorionic matrix comprises viable cells, wherein the composition does not comprise trophoblasts or an amniotic matrix.

In some aspects, minced chorionic matrix can comprise native, viable cells. In some aspects, the native, viable cells have not been culturally expanded. In some aspects, the native, viable cells have never been removed from the chorionic matrix. In some aspects, minced chorionic matrix is not substantially devitalized. Minced chorionic matrix can comprise some dead cells. In some aspects, minced chorionic matrix can comprise greater than 50%, 60%, 70%, 80%, 90%, 95% viable cells. In some aspects, the minced chorionic matrix can comprise greater than or equal to 100,000 viable cells/ml. In some aspects, the ratio of viable chorionic cells to all other nonviable cells in the composition can be 5:1, 2:1, 1:1, 1:2, 1:5, 1:10, 1:20, 1:30, 1:40, 1:50, 1:60, 1:70, 1:80, 1:90 or 1:100.

In some aspects, the homogenized UC matrix is devitalized. In some aspects, the homogenized UC matrix is not decellularized.

In some aspects, the minced chorionic matrix and homogenized UC matrix are from the same donor. In some aspects, the minced chorionic matrix and homogenized UC matrix are from different donors.

In some aspects, the disclosed compositions can comprise viable chorionic stem cells, fibroblasts, epithelial cells or a combination thereof.

In some aspects, the homogenized UC matrix comprises de-veined UC tissue.

4. Composition Comprising Isolated, Viable Chorionic Cells and a UC Matrix, Wherein the Composition Does Not Comprise Trophoblasts or an Amniotic Matrix

Disclosed are compositions comprising isolated, viable chorionic cells and a UC matrix, wherein the composition does not comprise trophoblasts or an amniotic matrix.

Disclosed are compositions comprising isolated, viable chorionic cells and a homogenized UC matrix, wherein the composition does not comprise trophoblasts or an amniotic matrix.

In some aspects, the UC matrix can be a non-homogenized UC matrix.

In some aspects, the disclosed compositions can comprise viable chorionic stem cells, fibroblasts, epithelial cells or a combination thereof.

In some aspects, the homogenized UC matrix comprises de-veined UC tissue.

5. Compositions With a Viscous Modifier

In some aspects, a viscous modifier can be added to any of the disclosed compositions. Thus, disclosed are compositions comprising a minced chorionic matrix, a homogenized amniotic matrix, a homogenized UC matrix and a viscous modifier, wherein the minced chorionic matrix comprises viable cells.

In some aspects, the viscous modifier can be hyaluronic acid, methylcellulose, carboxymethylcellulose, xanthum gum, pluronics, thermally responsive polymers (e.g. PNIPAAM) and proteins, fibronectins, laminins, collagens, chitosan, or chondroitin sulfate.

In some aspects, a viscous modifier allows or helps the disclosed compositions to be formulated as a cream, gel, oil, ointment, or lotion.

6. Compositions With a Scaffold

In some aspects, a scaffold can be added to any of the disclosed compositions. Thus, for example, disclosed are compositions comprising a minced chorionic matrix, a homogenized amniotic matrix, a homogenized UC matrix and a scaffold, wherein the minced chorionic matrix comprises viable cells.

In some aspects, the scaffold can be natural or synthetic. In some aspects, scaffold is a natural or synthetic polymer. In some aspects, the scaffold can be derived from skin, hyaline cartilage, meniscus, intervertebral disc, or bone. In some aspects, any type of tissue can be used as a scaffold. For example, tissue can be made into a matrix by mincing or homogenizing the tissue. In some aspects, placenta can be used as a scaffold.

In some aspects, a scaffold helps provide a matrix or structure for the disclosed compositions wherein the compositions can then be used in surgical applications. In some aspects, scaffolds can help give the compositions a specific shape.

7. Pharmaceutical Compositions

Disclosed are pharmaceutical compositions comprising any one of the compositions disclosed herein and a pharmaceutically acceptable carrier.

As disclosed herein, are pharmaceutical compositions, comprising the compositions disclosed herein. In an aspect, the pharmaceutical composition can comprise a minced chorionic matrix, a homogenized amniotic matrix and a devitalized homogenized umbilical cord (UC) matrix, wherein the non-homogenized chorionic matrix comprises viable cells, wherein the composition does not comprise trophoblasts. In an aspect, the pharmaceutical composition can comprise an isolated chorionic cells, a homogenized amniotic matrix and a devitalized homogenized umbilical cord (UC) matrix, wherein the composition does not comprise trophoblasts. In an aspect, the pharmaceutical composition can comprise a minced chorionic matrix and a devitalized homogenized umbilical cord (UC) matrix, wherein the non-homogenized chorionic matrix comprises viable cells, wherein the composition does not comprise trophoblasts or an amniotic matrix. In an aspect, the pharmaceutical composition can comprise isolated chorionic cells, a homogenized amniotic matrix and a devitalized homogenized umbilical cord (UC) matrix, wherein the composition does not comprise trophoblasts.

In some aspects, the pharmaceutical compositions can further comprise a pharmaceutically acceptable carrier. In some aspects, the pharmaceutical compositions described herein can be sterile and contain any of the disclosed compsotions for producing the desired response in a unit of weight or volume suitable for administration to a subject. In some aspects, the pharmaceutical compositions can contain suitable buffering agents, including, e.g., acetic acid in a salt; citric acid in a salt; boric acid in a salt; and phosphoric acid in a salt.

When administered, the composition can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term denotes an organic or inorganic ingredient, natural or synthetic, with which the active ingredient is combined to facilitate the application. The components of the pharmaceutical compositions also are capable of being co-mingled with the disclosed compositions, and with each other, in a manner such that there is no interaction which would substantially impair the desired pharmaceutical efficacy.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the compositions described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be any of the growth hormone releasing hormone peptides described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

In some aspects, administration of compositions disclosed herein can be administered to mammals other than humans, e.g., for testing purposes or veterinary therapeutic purposes, can be carried out under substantially the same conditions as described above.

8. Cryopreserved or Lyophilized Compositions

In some aspects, the disclosed compositions can be cryopreserved. In some aspects, the disclosed compositions can further comprise a cryopreservation solution. Thus, for example, disclosed are compositions comprising a minced chorionic matrix, a homogenized amniotic matrix and a homogenized UC matrix, wherein the minced chorionic matrix comprises viable cells and further comprising a cryopreservation solution. A further example includes, compositions comprising isolated, viable chorionic cells, a homogenized amniotic matrix, and a homogenized UC matrix, wherein the composition does not comprise trophoblasts, and further comprising a cryopreservation solution.

Disclosed herein are cryopreserved compositions comprising a minced chorionic matrix, a homogenized amniotic matrix, and a homogenized umbilical cord (UC) matrix, wherein the minced chorionic matrix comprises viable cells, wherein the composition does not comprise trophoblasts.

Disclosed herein are cryopreserved compositions comprising isolated, viable chorionic cells, a homogenized amniotic matrix, and a homogenized UC matrix, wherein the composition does not comprise trophoblasts.

Disclosed herein are cryopreserved compositions comprising a minced chorionic matrix and a homogenized UC matrix, wherein the minced chorionic matrix comprises viable cells, wherein the composition does not comprise trophoblasts or an amniotic matrix.

Disclosed herein are cryopreserved compositions comprising isolated, viable chorionic cells and a homogenized UC matrix, wherein the composition does not comprise trophoblasts or an amniotic matrix.

Disclosed herein are cryopreserved compositions comprising isolated, viable chorionic cells, a homogenized UC matrix, and a homogenized amniotic matrix wherein the composition does not comprise trophoblasts.

In some aspects, the cryopreserved compositions comprise at least 70% native, viable cells.

In some aspects, the cryoperserved composition, when thawed can comprise at least 70% viable cells. In some aspects, the cryoperserved or previously cryopreserved composition can comprise greater than 40%, 45%, 50%, 55%, 60%, 65%, 70%, 75%, 80%, 85%, 90%, 95%, or 99% viable cells. In some aspects, the cryoperserved or previously cryopreserved composition can then be cut to a desired size. Percent viability of cells after thawing is based on the percent of viable cells that were in the starting tissue sample prior to being frozen or cryopreserved.

In some aspects, a cryopreservation solution can contain one or more non-cell permeating cryopreservatives. Examples of non-cell permeating cryopreservatives, include but not limited to, polyvinyl pyrrolidione, a hydroxyethyl starch, a polysaccharide, a monosaccharide, an alginate, trehalose, raffinose, dextran, human serum albumin, Ficoll, lipoproteins, polyvinyl pyrrolidone, hydroxyethyl starch, autologous plasma or a mixture thereof. In some aspects, the cryopreservative does not contain DMSO or glycerol. Further, a cryopreservation solution can contain serum albumin or other suitable proteins to stabilize the disclosed compositions during the freeze-thaw process and to reduce the damage to cells, thereby maintaining viability. In some aspects, a cryopreservation solution can contain a physiological solution, such as a physiological buffer or saline, for example phosphate buffer saline. In some aspects, a cryopreservation solution can comprise a lyoprotectant, such as trehalose or trehalose in combination with one or more antioxidants.

In some aspects, the disclosed compositions can be lyophilized.

Disclosed are lyophilized tissues prepared using the disclosed methods of lyophilizing a tissue sample comprising obtaining a tissue sample, contacting the tissue sample with a lyoprotectant solution, freezing the tissue sample, performing a first drying step of the tissue sample after freezing, and performing a second drying step of the tissue sample after the first drying step.

Disclosed are lyophilized tissues prepared by a method comprising obtaining a tissue sample, contacting the tissue sample with a lyoprotectant solution, freezing the tissue sample, performing a first drying step of the tissue sample after freezing, performing a second drying step of the tissue sample after the first drying step and further comprising a step of reconstituting the lyophilized tissue.

Disclosed herein are lyophilized compositions comprising a minced chorionic matrix, a homogenized amniotic matrix, and a homogenized umbilical cord (UC) matrix, wherein the minced chorionic matrix comprises viable cells, wherein the composition does not comprise trophoblasts.

Disclosed herein are lyophilized compositions comprising isolated, viable chorionic cells, a homogenized amniotic matrix, and a homogenized UC matrix, wherein the composition does not comprise trophoblasts.

Disclosed herein are lyophilized compositions comprising a minced chorionic matrix and a homogenized UC matrix, wherein the minced chorionic matrix comprises viable cells, wherein the composition does not comprise trophoblasts or an amniotic matrix.

Disclosed herein are lyophilized compositions comprising isolated, viable chorionic cells and a homogenized UC matrix, wherein the composition does not comprise trophoblasts or an amniotic matrix.

Disclosed herein are lyophilized compositions comprising isolated, viable chorionic cells, a homogenized UC matrix, and a homogenized amniotic matrix wherein the composition does not comprise trophoblasts.

In some aspects, the disclosed lyophilized compositions comprise less than 15% residual water. In some aspects, the disclosed lyophilized compositions comprise 5-12% residual water. In some aspects, the disclosed lyophilized compositions comprise≤5% residual water.

In some aspects, the disclosed lyophilized compositions comprise trehalose. In some aspects, the disclosed lyophilized compositions comprise trehalose, wherein the trehalose is present at a concentration of 0.25M—1.5M.

In some aspects, the lyophilized composition, when reconstituted can comprise at least 70% viable cells. In some aspects, reconstituted tissue can comprise greater than 40%, 45%, 50%, 55%, 60%, 65%, 70%, 75%, 80%, 85%, 90%, 95%, or 99% viable cells. In some aspects, after reconstituting the lyophilized tissue, the tissue can then be cut to a desired size. Percent viability of cells after reconstitution is based on the percent of viable cells that were in the starting tissue sample prior to being lyophilized.

Disclosed are lyophilized compositions prepared using the methods disclosed herein that are sealed inside a sterile package.

In some aspects, the lyophilized compositions disclosed herein can be stable for at least three weeks. In some aspects, the lyophilized compositions can be stable for at least three months. In some aspects, the lyophilized compositions can be stable for 1, 2, 3, 4, 5, 6, 7, 8, 9, 10, 11, 12, 24, 36, 48, or 60 months.

In some aspects, the lyophilized compositions disclosed herein can be reconstituted resulting in a reconstituted tissue. The described lyophilized compositions can be reconstituted using standard techniques known in the art. In some aspects, reconstituting refers to rehydrating. Thus, the disclosed lyophilized compositions can be reconstituted or rehydrated using water, saline, a buffer such as, but not limited to phosphate buffered saline (PBS), in a solution comprising a stabilizing agent such as, but not limited to bovine serum albumin (BSA), Plasma-Lyte A or other clinically available electrolyte solutions, with human bodily fluids or a combination thereof. For example, lyophilized compositions can be applied directly to a wound or tissue injury on a subject and the subject's bodily fluids can reconstitute. In some aspects, a combination of bodily fluids and another known rehydrating solution can be used. Also, disclosed are reconstituted compositions prepared using the methods disclosed herein.

The reconstituted compositions derived from the methods disclosed herein can comprise native viable cells and native therapeutic factors. The reconstituted compositions can comprise at least 40%, 45%, 50%, 55%, 60%, 65%, 70%, 75%, 80%, 85%, 90%, 95%, 99% viable cells compared to the same compositions prior to lyophilization. The reconstituted compositions can comprise at least 40%, 45%, 50%, 55%, 60%, 65%, 70%, 75%, 80%, 85%, 90%, 95%, 99% viable native cells compared to the same compositions prior to lyophilization.

C. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for making the disclosed compositions, the kit comprising two or more of chrionic matrix, isolated chorionic cells, homogenized UC matrix and homogenized amniotic matrix.

In one aspect, disclosed are kits comprising a disclosed lyophilized composition and one or more of: (a) water, saline, or a buffer such as, but not limited to phosphate buffered saline (PBS), in a solution comprising a stabilizing agent such as, but not limited to bovine serum albumin (BSA), Plasma-Lyte A or other clinically available electrolyte solutions, with human bodily fluids or a combination thereof; and (b) instructions for reconstituting lyophilized tissue.

In various aspects, the informational material can be descriptive, instructional, marketing or other material that relates to the methods described herein and/or to the use of the lyophilized or reconstituted compositions for the methods described herein.

In various aspects, the composition of the kit can include other ingredients, such as a solvent or buffer, a stabilizer, a preservative, a fragrance or other cosmetic ingredient. In such aspects, the kit can include instructions for the lyophilized or reconstituted compositions and the other ingredients, or for using one or more compounds together with the other ingredients.

D. Methods of Making Compositions

Disclosed are methods of making one of the compositions disclosed herein comprising preparing a minced chorionic matrix; preparing a UC matrix; and combining the minced chorionic matrix and the UC matrix.

Disclosed are methods of making one of the compositions disclosed herein comprising preparing a minced chorionic matrix; preparing a homogenized UC matrix; and combining the minced chorionic matrix and the homogenized UC matrix.

Disclosed are methods of making one of the compositions disclosed herein comprising preparing isolated chorionic cells; preparing a homogenized UC matrix; combining the isolated chorionic cells and the homogenized UC matrix. In some aspects, the method can further comprise preparing a non-homogenized chorionic matrix.

In some aspects, the disclosed methods of making one of the compositions disclosed herein can further comprise lyophilizing the combined minced chorionic matrix and the homogenized UC matrix or the combined isolated chorionic cells and UC matrix.

Disclosed are methods of making the compositions disclosed herein comprising preparing a non-homogenized chorionic matrix, preparing a homogenized amniotic matrix, preparing a homogenized UC matrix, and combining the non-homogenized chorionic matrix, the homogenized amniotic matrix, and the homogenized UC matrix into a single composition. In some aspects, the methods of making the compositions disclosed herein further comprises combining viable, isolated amniotic cells to the composition.

Disclosed are methods of making the compositions disclosed herein comprising preparing a non-homogenized chorionic matrix, preparing a non-homogenized amniotic matrix, preparing a homogenized UC matrix, and combining the non-homogenized chorionic matrix, the non-homogenized amniotic matrix, and the homogenized UC matrix into a single composition.

Disclosed are methods of making the compositions disclosed herein comprising preparing a non-homogenized chorionic matrix, preparing a homogenized amniotic matrix, preparing a non-homogenized UC matrix, and combining the non-homogenized chorionic matrix, the homogenized amniotic matrix, and the non-homogenized UC matrix into a single composition.

Disclosed are methods of making one of the compositions disclosed herein comprising isolating chorionic tissue, isolating amniotic tissue, isolating and deveining UC tissue, rinsing each of the isolated chorionic tissue, isolated amniotic tissue, and deveined UC tissue individually, mincing or digesting the isolated chorionic tissue, combining and homogenizing the isolated amniotic tissue and the deveined UC tissue to form a placental matrix, and combining the minced or digested (i.e. non-homogenized) chorionic tissue with the placental matrix.

Disclosed are methods of making one of the compositions disclosed herein comprising isolating chorionic tissue, isolating amniotic tissue, isolating and deveining UC tissue, rinsing each of the isolated chorionic tissue, isolated amniotic tissue, and deveined UC tissue individually, mincing or digesting the isolated chorionic tissue, mincing or digesting the isolated amniotic tissue; homogenizing the deveined UC tissue; combining the minced or digested (i.e. non-homogenized) amniotic tissue and the homogenized UC tissue to form a placental matrix, and combining the minced or digested chorionic tissue with the placental matrix.

Disclosed are methods of making one of the compositions disclosed herein comprising isolating chorionic tissue, isolating amniotic tissue, isolating and deveining UC tissue, rinsing each of the isolated chorionic tissue, isolated amniotic tissue, and deveined UC tissue individually, mincing or digesting the isolated chorionic tissue, mincing or digesting the isolated amniotic tissue; mincing or digesting the deveined UC tissue; combining the non-homogenized amniotic tissue and the non-homogenized UC tissue to form a placental matrix, and combining the minced or digested chorionic tissue with the placental matrix.

Disclosed are methods of making one of the compositions disclosed herein comprising isolating chorionic tissue, isolating amniotic tissue, isolating and deveining UC tissue, rinsing each of the isolated chorionic tissue, isolated amniotic tissue, and deveined UC tissue individually, mincing or digesting the isolated chorionic tissue, homogenizing the isolated amniotic tissue; mincing or digesting the deveined UC tissue; combining the homogenized amniotic tissue and the non-homogenized UC tissue to form a placental matrix, and combining the minced or digested chorionic tissue with the placental matrix.

In some aspects, the disclosed methods of making one or more of the compositions disclosed herein can further comprise adding a viscous modifier. Any of the viscous modifiers disclosed herein can be used.

In some aspects, the disclosed methods of making one or more of the compositions disclosed herein can further comprise adding a scaffold. In some aspects, the scaffold is natural or synthetic. In some aspects, the scaffold can be derived from a meniscus, a disc, or bone. In some aspects, the scaffold can be a natural or synthetic polymer. Suitable scaffolds include, but are not limited to, for example, allografts, autografts, xenografts, ceramics, bioglass, calcium sulphate, demineralized bone matrix, coral, collagen, graft composites, chondronic scaffolds, synthetic scaffolds of all types, natural/biological scaffolds of all types and the like (e.g., calcium phosphates, hydroxyapatite and tricalcium phosphate, collagen/ceramic composite, PCL, PLLA,PLGA, PEG, PGA, alginates, silk, collagen, dextran gelatin, elastin, agarose, chitosan, hyaluronan, HA-TCP-Collagen, GraftJacket®, Alloderm®, PriMatrix® and others). Types thereof include, but are not limited to, other configurations such as sponges, foams, films, sheets, gels.

In some aspects, the chorionic matrix and UC matrix can be derived from the same donor. In some aspects, the chorionic matrix and amniotic matrix can be derived from the same donor. In some aspects, the amniotic matrix and UC matrix can be derived from the same donor. In some aspects, the chorionic matrix, amniotic matrix and UC matrix can be derived from the same donor. In some aspects, each of the chorionic matrix, amniotic matrix and UC matrix can be derived from different donors. In some aspects, at least one of the chorionic matrix, amniotic matrix and UC matrix is from a different donor than the other two.

In some aspects, the isolated chorionic cells and UC matrix can be derived from the same donor. In some aspects, the isolated chorionic cells and amniotic matrix can be derived from the same donor. In some aspects, the amniotic matrix and UC matrix can be derived from the same donor. In some aspects, the isolated chorionic cells, amniotic matrix and UC matrix can be derived from the same donor. In some aspects, each of the isolated chorionic cells, amniotic matrix and UC matrix can be derived from different donors. In some aspects, at least one of the isolated chorionic cells, amniotic matrix and UC matrix is from a different donor than the other two.

In some aspects, the methods of making the compositions disclosed herein further comprises combining viable, isolated amniotic cells to the composition.

1. Preparing Chorionic Matrix

In some aspects, preparing chorionic tissue comprises preparing homogenized, non-homogenized, or minced chorionic tissue.

In some aspects, preparing chorionic tissue can first comprise isolating chorionic tissue. Isolating chorionic tissue can be performed using techniques well known in the art. In some aspects, isolating chorionic tissue includes separating the chorion from the remaining placental tissue. Thus, in some aspects, chorionic tissue only comprises the chorion or portions thereof.

In some aspects, isolating chorionic tissue includes depleting the chorionic tissue of immunogenic cells and factors. This can be done using the methods described herein. In some aspects, immunogenic cells or factors can be removed from the non-homogenized chorionic matrix. In some aspects, non-homogenized chorionic matrix can be made immunocompatible by selectively depleting it of functional immunogenic cells. A chorion, chorionic tissue, or chorionic matrix can be made immunocompatible by selectively removing immunogenic cells from the chorion relative to therapeutic cells. For example, immunogenic cells can be removed by depleting or devitalizing the immunogenic cells or by purification of chorionic tissue there from.

In some aspects, chorionic matrix is gently prepared in order to preserve cell viability of the chorionic cells. Thus, the chorionic matrix is a non-homogenized chorionic matrix. In some aspects, preparing a non-homogenized chorionic matrix can comprise mincing, dicing, chopping, or digesting chorionic tissue to form a non-homogenized chorionic matrix. Preparing a chorionic matrix can result in the chorionic matrices disclosed herein. In some aspects, chorionic matrix comprises only chorionic tissue and no other placental tissue or UC tissue.

In some aspects, preparing a non-homogenized chorionic matrix comprises mincing chorionic tissue.

In some aspects, preparing a non-homogenized chorionic matrix comprises removing the trophoblast layer. Removal of the trophoblast layer can render the chorionic matrix immunocompatible.

In some aspects, the chorionic matrix can be made immunocompatible by selective depletion of functional+macrophages, optionally resulting in depleteion of TNFα upon stimulation, or a combination thereof.

In some aspects, the chorionic matrix can be made immunocompatible by selective depletion of maternal blood cells.

In some aspects, the chorionic matrix can be made immunocompatible by selective depletion of functional macrophages, trophoblasts, and vascularized tissue-derived cells.

In some aspects, the chorionic matrix can be made immunocompatible by selective depletion of trophoblasts and/or macrophages, optionally resulting in depletion of TNFα upon stimulation.

i. Trophoblast Removal

In some aspects, trophoblasts are selectively depleted or removed from the chorionic matrix. Surprisingly, trophoblast depleted chorionic tissue has one or more of the following superior features: is substantially non-immunogenic; and provides enhanced therapeutic efficacy.

Trophoblasts can be removed in any suitable manner which substantially diminishes the trophoblast content of the chorionic tissue. Optionally, the trophoblasts are selectively removed or otherwise removed without eliminating a substantial portion of one or more therapeutic components from the chorionic tissue (e.g. MSCs, chorionic factors, etc). Optionally, a majority (e.g. substantially all) of the trophoblasts are removed.

One method of removing trophoblasts comprises treating the chorionic tissue with a digestive enzyme such as dispase (e.g. dispase II) and separating the trophoblasts from the chorionic tissue. Optionally, the step of separating comprises mechanical separation such as peeling or scraping. Optionally, scraping comprises scraping with a soft instrument such as a finger.

One method of removing trophoblasts comprises treating the chorionic membrane with dispase for about 30 to about 45 minutes separating the trophoblasts from the chorionic tissue. Optionally, the dispase is provided in a solution of about less than about 1% (e.g. about 0.5%). Optionally, the step of separating comprises mechanical separation such as peeling or scraping. Optionally, scraping comprises scraping with a soft instrument such as a finger.

Useful methods of removing trophoblasts from a placenta (e.g. chorion) are described by Portmann-Lanz et al. (“Placental mesenchymal stem cells as potential autologous graft for pre- and perinatal neuroregeneration”; American Journal of Obstetrics and Gynecology (2006) 194, 664-73), (“Isolation and characterization of mesenchymal cells from human fetal membranes”; Journal Of Tissue Engineering And Regenerative Medicine 2007; 1: 296-305.), and (Concise Review: Isolation and Characterization of Cells from Human Term Placenta: Outcome of the First International Workshop on Placenta Derived Stem Cells”).

In some aspects, trophoblasts are removed before cryopreservation or lyophilization.

ii. Macrophage Depletion or Devitalization

In some aspects, functional macrophages are selectively depleted or devitalized from the chorionic tissue. Surprisingly, macrophage depleted chorionic tissue has one or more of the following superior features: is substantially non-immunogenic; provides remarkable healing time; and provides enhanced therapeutic efficacy.

Functional macrophages can be removed in any suitable manner which substantially diminishes the macrophage content of the chorionic tissue. Optionally, the macrophages are selectively depleted or devitalized without eliminating a substantial portion of one or more therapeutic components from the chorionic tissue (e.g. MSCs, chorionic factors, etc.). Optionally, a majority (e.g. substantially all) of the macrophages are depleted or devitalized.

One method of selectively depleting immune cells such as macrophages comprises depleting or devitalizing the immune cells by rapid freezing rates such as 60-100° C./min. Although immune cells can be eliminated by rapid freezing rates, such a method can also be detrimental to therapeutic cells such as stromal cells (e.g. MSCs). Disclosed is a method of selectively depleting or devitalizing macrophages by refrigerating the chorionic tissue for a period of time (e.g. for at least about 10 min such as for about 30-60 mins) at a temperature above freezing (e.g. incubating at 2-8° C.) and then freezing the chorionic tissue (e.g. incubating at −80° C.±5° C.). Optionally, the step of freezing comprises freezing at a rate of less than 10°/min (e.g. less than about 5°/min such as at about 1°/min).

In some aspects, the step of refrigerating comprises soaking the chorionic tissue in a cryopreservation medium for a period of time sufficient to allow the cryopreservation medium to penetrate (e.g. equilibrate with) the chorionic tissue. In some aspects, the cryopreservation solution used in the methods disclosed herein can comprise DMSO and/or glycerol. In some aspects, the cryopreservation solution does not comprise DMSO or glycerol. Optionally, the step of freezing comprises reducing the temperature at a rate of about 1°/min. Optionally, the step of freezing comprises freezing at a rate of less than 10°/min (e.g. less than about 5°/min such as at about 1°/min).

In some aspects, the step of refrigerating comprises soaking the chorionic tissue in a cryopreservation solution at a temperature of about −10-15° C. (e.g. at 2-8° C.) for at least about any of: 10 min, 20 min, 30 min, 40 min, or 50 min. In another embodiment, the step of refrigerating comprises soaking the chorionic tissue in a cryopreservation medium (e.g. containing DMSO) at a temperature of about −10-15° C. (e.g. at 2-8° C.) for about any of: 10-120, 20-90 min, or 30-60 min. Optionally, the step of freezing comprises freezing at a rate of less than 10°/min (e.g. less than about 5°/min such as at about 1°/min).

iii. Removal of Maternal Blood Cells

In some aspects, maternal blood cells from vascularized tissue are depleted or removed from the placental product. Surprisingly, chorionic tissue depleted of maternal blood cells has one or more of the following superior features: is substantially non-immunogenic; provides remarkable healing time; and provides enhanced therapeutic efficacy.

Maternal blood cells can be removed in any suitable manner which substantially diminishes such cell content of the chorionic tissue. Optionally, the maternal blood cells are selectively removed or otherwise removed without eliminating a substantial portion of one or more therapeutic components from the chorionic tissue (e.g. MSCs, chorionic factors, etc.).

In some aspects, removal of maternal blood cells comprises separating the chorion from the placenta by cutting around the placental skirt on the side opposite of the UC. The chorion on the umbilical side of the placenta is not removed due to the vascularization on this side.

In some aspects, removal of maternal blood cells comprises rinsing the chorionic membrane (e.g. with buffer such as PBS) to remove gross blood clots and any excess blood cells.

In some aspects, removal of maternal blood cells comprises treating the chorionic membrane with an anticoagulant (e.g. citrate dextrose solution).

In some aspects, removal of maternal blood cells comprises separating the chorion from the placenta by cutting around the placental skirt on the side opposite of the UC and rinsing the chorionic membrane (e.g. with buffer such as PBS) to remove gross blood clots and any excess blood cells.

In some aspects, removal of maternal blood cells comprises separating the chorion from the placenta by cutting around the placental skirt on the side opposite of the UC and treating the chorionic membrane with an anticoagulant (e.g. citrate dextrose solution).

In some aspects, removal of maternal blood cells comprises separating the chorion from the placenta by cutting around the placental skirt on the side opposite of the UC, rinsing the chorionic membrane (e.g. with buffer such as PBS) to remove gross blood clots and any excess blood cells, and treating the chorionic membrane with an anticoagulant (e.g. citrate dextrose solution).

2. Preparing Isolated Chorionic Cells

In some aspects, the disclosed methods of making the disclosed compositions can comprise preparing isolated chorionic cells comprises isolating chorionic cells from chorionic tissue.

In some aspects, chorionic cells can be isolated from chorionic tissue by either treating the chorionic tissue with enzymes to help release the cells or by homogenizing the tissue to help loosen the cells. In some aspects, the method can further comprise centrifugation.

In some aspects, preparing isolated chorionic cells comprises removing chorionic cells from chorionic tissue. In some aspects, the isolated cells can be washed. In some aspects, the cells can be cultured in cell culture media. In some aspects, the isolated cells are never cultured.

In some aspects, the isolated chorionic cells and UC matrix are derived from the same donor. In some aspects, the isolated chorionic cells and UC matrix are derived from different donors. In some aspects, the isolated chorionic cells and amniotic matrix are derived from the same donor. In some aspects, the isolated chorionic cells and amniotic matrix are derived from different donors. In some aspects, the isolated chorionic cells, UC matrix, and amniotic matrix are all from the same donor. In some aspects, at least one of the isolated chorionic cells, UC matrix, and amniotic matrix is from a different donor compared to the other two.

3. Preparing UC Matrix

UC matrix for use in the disclosed methods and compositions can be prepared by homogenization or non-homogenization. Homogenization can include, but is not limited to, those techniques that make the UC tissue uniform and identical throughout. Thus, homogenization can include blending, crushing dried or frozen tissue using a mortar and pestle, milling at room temperature, milling while frozen, and using a tissue homogenizer. In some aspects, homogenized UC matrix is not decellularized. In some aspects, the homogenized UC matrix can be devitalized. Preparing an UC matrix can result in the UC matrices disclosed herein. In some aspects, UC matrix comprises only UC tissue and no placental tissue.

In some aspects, the homogenized or non-homogenized UC matrix can comprise de-veined UC tissue. De-veining UC can be performed using techniques well known in the art. For example, an UC can be slit or cut longitudinally using, e.g., a scalpel and forceps, grooved director, or the like. This allows the UC membrane to be laid flat, allowing, e.g., removal of the Wharton's jelly, and/or one or more of the UC arteries, veins e.g., with a forceps. The UC membrane can also be processed further without cutting and opening the membrane. An UC vessel, for example, can be removed from the cord by grasping the vessels with a forceps and gently pulling and massaging until the vessel is removed, leaving the UC membrane as an intact tube. In a preferred embodiment of deveining, the umbilical vein of an UC can be canalized using the blunt probe of a vein stripper. The blunt probe can be replaced with a small bullet probe, and the vein can be tied to the probe with thread. The stripper can then be removed, and the process can be repeated with the umbilical arteries.

In some aspects, the UC tissue for use in the disclosed methods and compositions is not homogenized and therefore can be a non-homogenized UC matrix. In some aspects, preparing a non-homogenized UC matrix can comprise mincing, dicing, chopping or digesting UC tissue to form a non-homogenized UC matrix.

In some aspects, a UC matrix for use in the disclosed methods and compositions can be, but is not limited to, cut into pieces, pre-chilled, added to chilled-solution, dried in oven before cryomilling, etc. In some aspects, the UC matrix can be cut using scissors or minced into smaller pieces. In some aspects, the UC matrix can be cut using scissors or minced into smaller pieces prior to homogenizing (e.g. blending or milling).

In some aspects, during blending, the tissue (e.g. the UC or amniotic tissue) can be submerged in chilled saline or PBS to maintain cool temperatures during homogenization. If milling, the UC matrix or amniotic matrix can be dried prior to cutting/mincing or after cutting/mincing. In some aspects, drying can be done at room temperature, using a warm oven, or using a lyophilizer (no freezing required). In some aspects, once dried, the tissue can be placed into a milling device (either a grinding mill or a ball-bearing based mill, or other such mill) to be ground into small particles and homogenized. If cryomilling, the tissue can be dried or not dried first, frozen by storage in a freezer overnight (slow freeze) or by application of liquid nitrogen (flash freeze), and then milled in a cooled chamber.

In some aspects, an amniotic tissue and UC tissue can be combined as whole cord and membrane or as pre-cut/minced pieces prior to homogenization.

4. Combining Chorionic Matrix and UC Matrix

In some aspects, any of the disclosed chorionic matrices can be combined with any of the disclosed UC matrices.

In some aspects, minced chorionic matrix can be combined with the disclosed UC matrix. In some aspects, minced chorionic matrix can be combined with the disclosed UC matrix and then that combination can be combined with a homogenized amniotic matrix.

In some aspects, minced chorionic matrix can be combined with a mixture of a homogenized amniotic matrix and a homogenized UC matrix. In some aspects, non-homogenized chorionic matrix can be combined with a homogenized amniotic matrix and then that combination can be combined with the homogenized UC matrix. In some aspects, non-homogenized chorionic matrix can be combined with the homogenized UC matrix and then that combination can be combined with the homogenized amniotic matrix. In some aspects, all of the non-homogenized chorionic matrix, homogenized amniotic matrix, and homogenized UC matrix can be combined simultaneously.

5. Combining Isolated Chorionic Cells and Homogenized UC Matrix

In some aspects, any of the disclosed isolated chorionic cells can be combined with any of the disclosed UC matrices.

In some aspects, isolated chorionic cells can be combined with the disclosed UC matrix. In some aspects, isolated chorionic cells can be combined with the disclosed UC matrix and then that combination can be combined with a homogenized amniotic matrix.

6. Preparing an Amniotic Matrix

In some aspects, preparing amniotic matrix comprises preparing homogenized or non-homogenized amniotic matrix.

In some aspects, the disclosed methods can further comprise preparing a homogenized amniotic matrix and combining the homogenized amniotic matrix with the minced chorionic matrix and the homogenized UC matrix.

In some aspects, the disclosed methods can further comprise the step of preparing a homogenized amniotic matrix and combining with the isolated chorionic cells and the homogenized UC matrix.

In some aspects, the disclosed methods can further comprise prior to preparing a homogenized amniotic matrix, isolating epithelial cells from the amniotic matrix. In some aspects, the disclosed methods can further comprise combining the isolated amniotic epithelial cells to the combined isolated chorionic cells, the homogenized amniotic matrix, and the homogenized UC matrix.

Amniotic matrix for use in the disclosed methods and compositions can be prepared by homogenization. Homogenization can include, but is not limited to, those techniques that make the amniotic tissue uniform and identical throughout. Thus, homogenization can include blending, crushing dried or frozen tissue using a mortar and pestle, milling at room temperature, milling while frozen (a.k.a cryomilling), and using a tissue homogenizer. In some aspects, homogenized amniotic matrix is not decellularized. In some aspects, the homogenized amniotic matrix can be devitalized. Preparing an amniotic matrix can result in the amniotic matrices disclosed herein. In some aspects, amniotic matrix comprises only amniotic tissue and no other placental tissue or UC tissue.

In some aspects, the amniotic tissue for use in the disclosed methods and compositions is not homogenized and therefore can be a non-homogenized amniotic matrix. In some aspects, preparing a non-homogenized amniotic matrix can comprise mincing, dicing, chopping or digesting amniotic tissue to form a non-homogenized amniotic matrix.

In some aspects, homogenized or non-homogenized amniotic matrix can be made immunocompatible by selectively depleting it of functional immunogenic cells. An amnion, amniotic tissue, or amniotic matrix can be made immunocompatible by selectively removing immunogenic cells from the amnion relative to therapeutic cells. For example, immunogenic cells can be removed by depleting or devitalizing the immunogenic cells or by purification of amniotic tissue there from. In some aspects, making the amniotic tissue immunocompatible by selectively depleting it of functional immunogenic cells can be performed by making sure the amnion is isolated from the remaining placental tissue. The removal of trophoblasts can be done by the methods described herein with regard to chorionic tissue or simply making sure the amnion is not associated with the chorion which comprises the trophoblast layer.

In some aspects, the selective depletion or devitalization of macrophages can be done by the methods described herein with regard to chorionic tissue.

In some aspects, the removal of maternal blood cells can be done by the methods described herein with regard to chorionic tissue.

In some aspects, the amniotic matrix can be combined with the chorionic matrix and UC matrix or combined with the isolated chorionic cells and UC matrix.

7. Combining Amniotic Tissue and UC Tissue to Form a Placental Matrix

In some aspects, isolated amniotic tissue and UC tissue can be homogenized to form an amniotic matrix and UC matrix, respectively. In some aspects, the amniotic tissue and UC tissue can be combined and then homogenized together to form a placental matrix. In some aspects, the amniotic tissue is homogenized to an amniotic matrix and the UC tissue is homogenized to an UC matrix and then the matrices are combined together to form a placental matrix. Thus, a placental matrix can be the combination of homogenized amniotic tissue and UC tissue.

In some aspects, homogenizing the isolated amniotic tissue and the deveined UC tissue to form a placental matrix comprises blending or milling the amniotic tissue and the deveined UC tissue together. In some aspects, any known homogenization technique can be used.

In some aspects, one or both of the isolated amniotic tissue and UC tissue can be non-homogenized to form an amniotic matrix and UC matrix, respectively.

In some aspects, the amniotic tissue and UC tissue can be combined and then homogenized together to form a placental matrix. In some aspects, the amniotic tissue can be minced, diced, chopped or digest into an amniotic matrix and the UC tissue can be homogenized to an UC matrix and then the matrices can be combined together to form a placental matrix. In some aspects, the amniotic tissue can be homogenized to an amniotic matrix and the UC tissue can be minced, diced, chopped or digest into to an UC matrix and then the matrices are combined together to form a placental matrix. In some aspects, a placental matrix can be the combination of non-homogenized amniotic tissue and homogenized UC tissue, homogenized amniotic tissue and non-homogenized UC tissue, or non-homogenized amniotic tissue and non-homogenized UC tissue.

In some aspects, the placental matrix can comprise viable and dead cells. In some aspects, the placental matrix is not decellularized. In some aspects, less than 50%, 40%, 30%, 20%, 10%, or 5% of the cells are viable in the placental matrix.

8. Combining the Minced or Digested Chorionic Tissue With the Placental Matrix

In some aspects, the minced or digested chorionic tissue can be combined with the placental matrix. In some aspects, the combined minced or digested chorionic tissue with the placental matrix can comprise viable chorionic cells. In some aspects, 50%, 60%, 70%, 80%, 90% or 95% of the viable cells are native chorionic cells.

9. Isolating Amniotic Cells

In some aspects, the disclosed methods of making the disclosed compositions can further comprise, prior to preparing a homogenized or non-homogenized amniotic matrix, performing the step of isolating amniotic cells from the amniotic matrix.

Disclosed are methods of making the compositions disclosed herein comprising preparing a non-homogenized chorionic matrix, preparing a homogenized amniotic matrix, preparing a homogenized UC matrix, and combining the non-homogenized chorionic matrix, the non-homogenized chorionic matrix, and the homogenized UC matrix further comprising prior to preparing a homogenized amniotic matrix, performing the step of isolating amniotic cells from the amniotic matrix. In some aspects, the non-homogenized chorionic matrix can be minced.

Disclosed are methods of making the compositions disclosed herein comprising preparing a non-homogenized chorionic matrix, preparing a homogenized amniotic matrix, preparing a homogenized UC matrix, and combining the non-homogenized chorionic matrix, the homogenized amniotic matrix, and the homogenized UC matrix further comprising prior to preparing a homogenized amniotic matrix, performing the step of isolating amniotic cells from the amniotic matrix and further comprising combining the isolated amniotic cells to the combined non-homogenized chorionic matrix, the homogenized amniotic matrix, and the homogenized UC matrix. In some aspects, the non-homogenized chorionic matrix can be minced.

Thus, for example, disclosed are methods of making the compositions disclosed herein comprising preparing a non-homogenized chorionic matrix, isolating amniotic cells from an amniotic tissue sample, preparing a homogenized amniotic matrix, preparing a homogenized UC matrix, and combining the non-homogenized chorionic matrix, the homogenized amniotic matrix, and the homogenized UC matrix.

Disclosed are methods of making the compositions disclosed herein comprising preparing a minced chorionic matrix, preparing a homogenized amniotic matrix, preparing a homogenized UC matrix, and combining the minced chorionic matrix, the homogenized amniotic matrix, and the homogenized UC matrix and further comprising, prior to preparing a homogenized amniotic matrix, performing the step of isolating amniotic cells from the amniotic matrix. In some aspects of the disclosed methods it can be unnecessary to isolate amniotic cells from the amniotic tissue prior to preparing a homogenized amniotic matrix. In some aspects, the disclosed methods can further comprise combining the isolated amniotic cells to the combined minced chorionic matrix, the homogenized amniotic matrix, and the homogenized UC matrix.

Disclosed are methods of making the compositions disclosed herein comprising preparing a minced chorionic matrix, preparing a homogenized UC matrix, and combining the minced chorionic matrix, and the homogenized UC matrix and further comprising, performing the step of isolating amniotic cells from an amniotic matrix. In some aspects, the disclosed methods can further comprise combining the isolated amniotic cells to the combined minced chorionic matrix and the homogenized UC matrix.

Disclosed are methods of making the compositions disclosed herein comprising preparing isolated chorionic cells, preparing a homogenized amniotic matrix, preparing a homogenized UC matrix, and combining the isolated chorionic cells, the homogenized amniotic matrix, and the homogenized UC matrix and further comprising, prior to preparing a homogenized amniotic matrix, performing the step of isolating amniotic cells from the amniotic matrix. In some aspects of the disclosed methods it can be unnecessary to isolate amniotic cells from the amniotic tissue prior to preparing a homogenized amniotic matrix. In some aspects, the disclosed methods can further comprise combining the isolated amniotic cells to the combined isolated chorionic cells, the homogenized amniotic matrix, and the homogenized UC matrix.

Disclosed are methods of making the compositions disclosed herein comprising preparing isolated chorionic cells, preparing a homogenized UC matrix, and combining the isolated chorionic cells and the homogenized UC matrix and further comprising, performing the step of isolating amniotic cells from an amniotic matrix. In some aspects, the disclosed methods can further comprise combining the isolated amniotic cells to the combined isolated chorionic cells and the homogenized UC matrix.

Disclosed are methods of making the compositions disclosed herein comprising preparing a non-homogenized chorionic matrix, preparing a non-homogenized amniotic matrix, preparing a homogenized UC matrix, and combining the non-homogenized chorionic matrix, the non-homogenized amniotic matrix, and the homogenized UC matrix and further comprising, prior to preparing a non-homogenized amniotic matrix, performing the step of isolating amniotic cells from the amniotic matrix. In some aspects of the disclosed methods it can be unnecessary to isolate amniotic cells from the amniotic tissue prior to preparing a non-homogenized amniotic matrix. In some aspects, the disclosed methods can further comprise combining the isolated amniotic cells to the combined non-homogenized chorionic matrix, the non-homogenized amniotic matrix, and the homogenized UC matrix.

Disclosed are methods of making the compositions disclosed herein comprising preparing a non-homogenized chorionic matrix, preparing a non-homogenized amniotic matrix, preparing a non-homogenized UC matrix, and combining the non-homogenized chorionic matrix, the non-homogenized amniotic matrix, and the non-homogenized UC matrix and further comprising, prior to preparing a non-homogenized amniotic matrix, performing the step of isolating amniotic cells from the amniotic matrix. In some aspects of the disclosed methods it can be unnecessary to isolate amniotic cells from the amniotic tissue prior to preparing a non-homogenized amniotic matrix. In some aspects, the disclosed methods can further comprise combining the isolated amniotic cells to the combined non-homogenized chorionic matrix, the non-homogenized amniotic matrix, and the non-homogenized UC matrix.

Disclosed are methods of making the compositions disclosed herein comprising preparing a non-homogenized chorionic matrix, preparing a homogenized amniotic matrix, preparing a non-homogenized UC matrix, and combining the non-homogenized chorionic matrix, the homogenized amniotic matrix, and the non-homogenized UC matrix and further comprising, prior to preparing a non-homogenized amniotic matrix, performing the step of isolating amniotic cells from the amniotic matrix. In some aspects, the disclosed methods can further comprise combining the isolated amniotic cells to the combined non-homogenized chorionic matrix, the homogenized amniotic matrix, and the non-homogenized UC matrix.

In some aspects, the isolated amniotic cells from amniotic tissue are isolated from the same amniotic tissue used to prepare a homogenized or non-homogenized amniotic matrix. In some aspects, the isolated amniotic cells from amniotic tissue are isolated from a different amniotic tissue used to prepare a homogenized or non-homogenized amniotic matrix.

In some aspects, amniotic cells can be isolated from amniotic tissue using known enzymatic or mechanical methods, such as treatment with an enzyme solution and/or mechanical scraping of the epithelial surface using commercially available cell scrapers. For example, amniotic stromal cells, either fibroblasts or MSCs, can be isolated from amniotic tissue using known enzymatic or mechanical methods.

In some aspects, isolated epithelial or stromal cells from amniotic tissue can be present in small clusters of two or more cells still connected by cell-cell junction proteins or extracellular matrix proteins, or as single cells.

The viable, isolated amniotic cells from amniotic tissue can be added to the non-homogenized chorionic matrix.

10. Isolating Epithelial Cells

In some aspects, the disclosed methods of making the disclosed compositions can further comprise, prior to preparing a homogenized or non-homogenized amniotic matrix, performing the step of isolating amniotic cells from the amniotic matrix.

Disclosed are methods of making one of the compositions disclosed herein comprising isolating chorionic tissue, isolating amniotic tissue, isolating and deveining UC tissue, rinsing each of the isolated chorionic tissue, isolated amniotic tissue, and deveined UC tissue individually, mincing or digesting the isolated chorionic tissue, combining and homogenizing the isolated amniotic tissue and the deveined UC tissue to form a placental matrix, and combining the minced or digested chorionic tissue with the placental matrix, further comprising isolating amniotic cells from the amniotic tissue prior to combining and homogenizing the isolated amniotic tissue and the deveined UC tissue to form a placental matrix and combining the isolated amniotic cells. In some aspects, the combining the isolated amniotic cells can occur after combining and homogenizing the isolated amniotic tissue and the deveined UC tissue to form a placental matrix. In some aspects, the combining the isolated amniotic cells can occur to the minced or digested chorionic tissue before combining the chorionic tissue with the placental matrix.

Disclosed are methods of making one of the compositions disclosed herein comprising isolating chorionic tissue, isolating amniotic tissue, isolating and deveining UC tissue, rinsing each of the isolated chorionic tissue, isolated amniotic tissue, and deveined UC tissue individually, mincing or digesting the isolated chorionic tissue, mincing or digesting the isolated amniotic tissue, homogenizing the deveined UC tissue, combining the non-homogenized amniotic tissue and homogenized UC tissue to form a placental matrix, and combining the minced or digested chorionic tissue with the placental matrix, further comprising isolating amniotic cells from the amniotic tissue prior to mincing or digesting the amniotic tissue and combining the isolated amniotic cells. In some aspects, the combining the isolated amniotic cells can occur after combining the non-homogenized amniotic tissue and homogenized UC tissue to form a placental matrix. In some aspects, the combining the isolated amniotic cells can occur to the minced or digested chorionic tissue before combining the chorionic tissue with the placental matrix.

Disclosed are methods of making one of the compositions disclosed herein comprising isolating chorionic tissue, isolating amniotic tissue, isolating and deveining UC tissue, rinsing each of the isolated chorionic tissue, isolated amniotic tissue, and deveined UC tissue individually, mincing or digesting the isolated chorionic tissue, combining and then mincing or digesting the isolated amniotic tissue and the deveined UC tissue to form a placental matrix, and combining the minced or digested chorionic tissue with the placental matrix, further comprising isolating amniotic cells from the amniotic tissue prior to combining and mincing or digesting the amniotic tissue and UC tissue to form a placental matrix and combining the isolated amniotic cells. In some aspects, the combining the isolated amniotic cells can occur after combining and mincing or digesting the isolated amniotic tissue and the deveined UC tissue to form a placental matrix. In some aspects, the combining the isolated amniotic cells can occur to the minced or digested chorionic tissue before combining the chorionic tissue with the placental matrix.

Disclosed are methods of making one of the compositions disclosed herein comprising isolating chorionic tissue, isolating amniotic tissue, isolating and deveining UC tissue, rinsing each of the isolated chorionic tissue, isolated amniotic tissue, and deveined UC tissue individually, mincing or digesting the isolated chorionic tissue, homogenizing the isolated amniotic tissue, mincing or digesting the deveined UC tissue, combining the homogenized amniotic tissue and non-homogenized UC tissue to form a placental matrix, and combining the minced or digested chorionic tissue with the placental matrix, further comprising isolating amniotic cells from the amniotic tissue prior to homogenizing the amniotic tissue and combining the isolated amniotic cells. In some aspects, the combining the isolated amniotic cells can occur after combining the homogenized amniotic tissue and non-homogenized UC tissue to form a placental matrix. In some aspects, the combining the isolated amniotic cells can occur to the minced or digested chorionic tissue before combining the chorionic tissue with the placental matrix.

11. Rinsing

Each of the individual tissues (i.e. chorionic, amniotic, UC) can be rinsed. In some aspects, rinsing can include rinsing with a saline solution. In some aspects, rinsing can include a red cell lysis solution. In some aspect, rinsing can include a solution with an antibiotic.

In some aspects, rinsing can be for the sole purpose of cleaning each of the tissues and removing any excess components that are not part of the specific tissue sample. For example, rinsing can remove blood clots.

12. Lyophilizing

The disclosed methods of making a composition comprising a chorionic matrix and a homogenized or non-homogenized UC matrix, wherein the chorionic matrix comprises viable cells can further comprise lyophilizing the combined chorionic matrix and UC matrix.

The disclosed methods of making a composition comprising a chorionic matrix and a homogenized or non-homogenized UC matrix, and an amniotic matrix, wherein the chorionic matrix comprises viable cells can further comprise lyophilizing the combined chorionic matrix, amniotic matrix, and UC matrix.

The disclosed methods of making a composition comprising isolated chorionic cells and a homogenized or non-homogenized UC matrix can further comprise lyophilizing the combined isolated chorionic cells and UC matrix.

The disclosed methods of making a composition comprising isolated chorionic cells, an amniotic matrix, and a homogenized or non-homogenized UC matrix can further comprise lyophilizing the combined isolated chorionic cells, amniotic matrix, and UC matrix.

The disclosed methods of making a composition comprising a non-homogenized chorionic matrix, a homogenized or non-homogenized amniotic matrix and a homogenized or non-homogenized UC matrix, wherein the non-homogenized chorionic matrix comprises viable cells can further comprise lyophilizing the combined chorionic tissue and placental matrix.

In some aspects, each of the components of the disclosed compositions can be lyophilized separately and then mixed together. In some aspects, one or more of the components of the disclosed compositions can be lyophilized together. In some aspects, each of the non-homogenized chorionic matrix, homogenized or non-homogenized amniotic matrix, homogenized or non-homogenized UC matrix and isolated amniotic cells can be lyophilized separately. In some aspects, after lyophilizing each component separately, each can then be combined together.

Any known lyophilization technique and equipment can be used. In some aspects, methods of lyophilizing the disclosed compositions can comprise contacting one of the disclosed compositions with a lyoprotectant solution, freezing the composition, performing a first drying step of the composition after freezing, and performing a second drying step of the composition after the first drying step.

In some aspects, methods of lyophilizing the disclosed compositions can comprise contacting one of the disclosed compositions with a lyoprotectant solution, freezing the composition, performing a first drying step of the composition after freezing, and performing a second drying step of the composition after the first drying step, and further comprising a step of reconstituting the lyophilized tissue.

In some aspects, contacting the composition with a lyoprotectant solution can include a short or prolonged contact. In some aspects, the first drying step of the composition after freezing occurs between −45° C. and −15° C. In some aspects, the second drying step can be carried out at a temperature that is greater than the temperature of the freezing step. In some aspects, the second drying step can be carried out at a temperature that is greater than the temperature of the freezing step and the first drying step.

In some aspects, contacting the composition with a lyoprotectant solution can include a short or prolonged contact. For example, the composition can be exposed or contacted to a lyoprotectant solution for 1, 2, 3, 4, 5, 10, 15, 20, 25, 30, 35, 40, 45, 50, 55, or 60 minutes. In some aspects, the composition can be exposed or contacted to a lyoprotectant solution for 1, 2, 3, 4, 5, 6, 7, 8, 9, 10, 12, 14, 16, 18, 20, 22, or 24 hours. In some aspects, the composition can be exposed or contacted to a lyoprotectant solution for 1, 2, 3, 4, 5, 6, 7, 14, 21 days. In some aspects, the composition can be exposed or contacted to a lyoprotectant solution for 1, 2, 3, 4, 5, 6, 7, or 8 weeks.

In some aspects, contacting the composition with a lyoprotectant solution can be the same as exposing the composition to a lyoprotectant solution or soaking the tissue sample in a lyoprotectant solution.

As described here, a lyoprotectant solution comprises at least one lyoprotectant. In some aspects, a lyoprotectant solution can comprise trehalose. Other lyoprotectants can include but are not limited to polyhydroxy compounds such as sugars, polyalcohols, raffinose, and other non-reducing polysaccharides, and their derivatives.

In some aspects, the lyoprotectant solution can further comprise one or more antioxidants. In some aspects, the one or more antioxidants can be epigallocatechin gallate (EGCG) or catechin. In some aspects, an antioxidant can be ascorbic acid, L-carnosine, spermine, phloretine, α-tocopherol, β-carotene, conenzyme Q10, lutein, melatonin, butylated hydroxytoluene, γ-tocopherol, lutein, N-acetyl-L-cysteine, mitoquinone, hydroquinone, lipoic acid, glutathione, carotenoids, polyphenols, retinol, tocotrienol.

In some aspects, lyoprotectant solution can also comprise saline, DMSO, antibiotics, bulking agents, excipients, or a combination thereof. In some aspects, the lyoprotectant can comprise other reagents that can improve lyophilization performance.

The concentration of a lyoprotectant or antioxidants present in the lyoprotectant solution and the length of time for contacting the tissue sample with the lyoprotectant solution can be dependent on the type and size of the tissue sample. Based on the teachings herein, one of skill in the art using routine methods would understand how to adjust the concentrations and contacting times.

In some aspects, contacting the tissue sample with a lyoprotectant solution can occur at temperatures between 0° and 39° C. In some aspects, contacting the tissue sample with a lyoprotectant solution can occur at 4° C.

E. Methods of Treating

Disclosed are methods of treating a tissue injury or chronic pain comprising administering one or more of the disclosed compositions to an area of a subject comprising a tissue injury. In some aspects, the tissue injury can be the tissue injury is osteoarthritis, cartilage repair, meniscus repair, intervertebral disc repair, plantar fasciitis, carpal tunnel, tendonitis, synovitis, ruptured or torn Achilles tendon, critical limb ischemia, ulcers, pyoderma gangrenosum, epidermolysis bullosa, surgical adhesions, plastic surgery, surgical applications, or other wounds. Also disclosed are methods of treating a subject comprising administering one or more of the disclosed compositions to a subject comprising dysphagia, vocal cord paralysis, osteonecrosis, neuroischemic wounds, ENT fistulas, or anal fistulas.

In some aspects, any of the disclosed compositions can be administered by injecting the composition to the area of a subject comprising a tissue injury or local region of pain or inflammation.

In some aspects, any of the disclosed compositions can be administered by applying the composition topically to an area of a subject comprising the tissue injury or pain or inflammation.

In some aspects, any of the disclosed compositions can be administered by implanting the composition to the area of a subject comprising a tissue injury.

In some aspects, the subject can be a mammal. In some aspects, the subject can be human.

### EXAMPLES

### A. Example 1

### A Composition Comprising a Minced Chorionic Matrix, a Homogenized Amniotic Matrix, and a Homogenized Umbilical Cord (UC) Matrix, Wherein the Minced Chorionic Matrix Comprises Viable Cells

1. Characterization of Amnion, Chorion, and Umbilical Cord.

The presence of angiogenic and other growth factors in amnion, chorion, and umbilical cord lysates was determined by multiplex assays (Luminex). Protein estimation was quantified by BCA assay and values were reported as picogram of target per milligram of total protein (Table 1).

TABLE 1

| | CM/AM/UC |
|---|---|
| Target | pg/mg |
| Angiogenin | 20007.5 |
| BMP-7 | 550.7 |
| FGF-23 | 27.6 |
| PDGF-AA | 119.1 |
| TIMP-1 | 30590 |
| VEGF-C | 268 |
| BMP-4 | 57.1 |
| FGF basic | 1907.4 |
| Osteopontin | 28482.7 |
| PDGF-BB | 191.5 |
| VEGF-A | 78.4 |
| IFN-gamma | 207.6 |
| IL-2 | 581.5 |
| IL-6 | 4038 |
| TNF-alpha | 38 |
| IL-1 beta | 1107.9 |
| IL-5 | 5.1 |
| CCL2/MCP-1 | 3632.1 |
| IL-1ra | 3523.3 |

2. Inhibition of TNF-α Secretion by Activated THP-1 Cells.

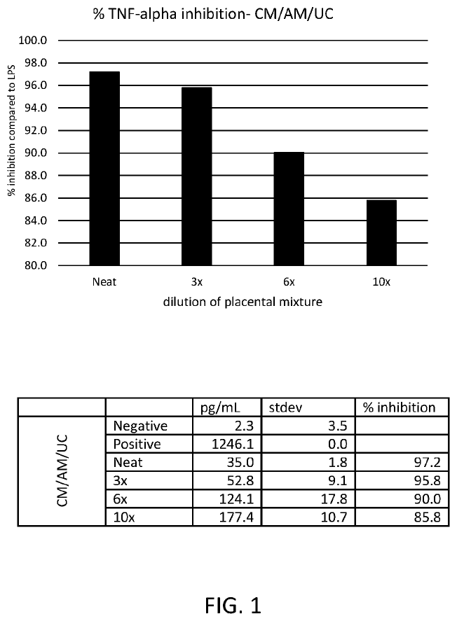

The anti-inflammatory capabilities of a composition comprising a minced chorionic matrix, a homogenized amniotic matrix, and a homogenized UC matrix, wherein the minced chorionic matrix comprises viable cells, and depleted of trophoblasts were characterized by the inhibition of TNFα produced by Lipopolysaccharide (LPS) activated THP-1 cells. THP-1 cells were plated in a 24 well plate and stimulated with lug/mL of LPS in DMEM growth media and co-cultured neat or with varying dilutions (3×, 6×, 10×) of minced chorion, amnion and umbilical cord (CM+AM+UC). Untreated THP-1 and LPS treated THP-1 cells represented positive and negative controls respectively. The culture was incubated overnight at 37° C. with 5% CO₂. After overnight stimulation and treatment, the cell media was collected and the amount of TNFα produced by the THP-1 cells after LPS stimulation is compared to the CM/AM/UC treatments and reported as % inhibition vs the positive control (FIG. 1). TNFα is quantified by Luminex and/or ELISA (R&D Systems).

THP1 cells are representatives of the inflammatory cells that infiltrate into the site of injury. These cells produce inflammatory cytokines like TNFα when triggered by stimulus, i.e. LPS. LPS represents an infectious component, commonly seen in an injured tissue. In the experiment, the positive control, TNFα released by THP1 cells upon LPS treatment, is representative of the TNFα present in an inflammatory microenvironment. Upon addition of CM/AM/UC, the level of TNFα response is diminished and this is quantified as percent inhibition vs control. The objective is to decrease TNFα levels upon treatment.

3. TSG-6 Levels Secreted by Fresh Minced Chorion Tissue.

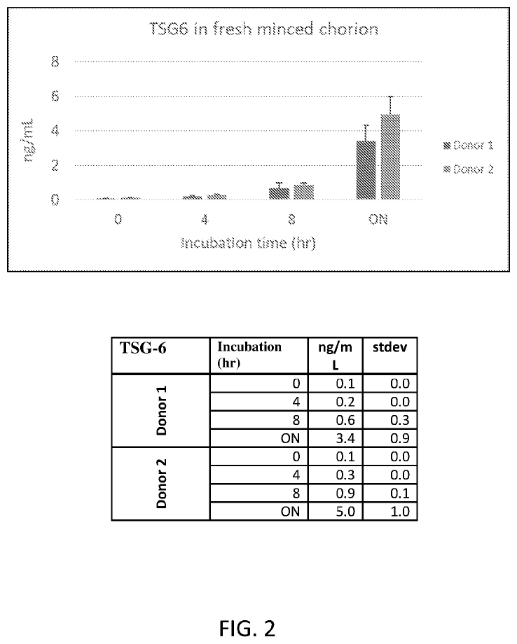

Fresh minced chorion was incubated for 0 hours, 4 hours, 8 hours, and overnight at 37° C. 5% CO2 in DMEM supplemented with 10% Fetal Bovine Serum 1% Penicillin Streptomycin. The presence of TSG-6, an anti-inflammatory protein, in conditioned media is determined by ELISA. (FIG. 2) TSG-6 is a multi-functional protein that is associated with inflammation and is upregulated in response to an inflammatory milieu. It has anti-inflammatory and chondroprotective effects, hence is capable of downregulating the inflammatory response. Higher levels of TSG-6 are indicative of the better resolution of inflammation.

In some aspects, fresh minced chorion acts in the same manner as whole chorion that has not been minced.

### B. Example 2

### A Composition Comprising an Isolated Chorionic Cells (CM Cells), a Homogenized Amniotic Matrix (AM) and a Devitalized Homogenized Umbilical Cord (UC) Matrix

1. Characterization of Chorion Cells and Amnion and Umbilical Cord Matrix.

The presence of angiogenic and other growth factors in chorion cells, amnion and umbilical cord lysates was determined by multiplex assays (Luminex). Protein estimation was quantified by BCA assay and values were reported as picogram of target per milligram total protein (Table 2).

TABLE 2

| | CM cells AM/UC |
|---|---|
| Target | pg/mg |
| IL-10 | 3.9 |
| FGF basic | 390.1 |
| Angiogenin | 2998.4 |
| Angiopoietin | 32.4 |
| HGF | 309.1 |
| PDGF-AA | 6.2 |
| PDGF-BB | 14.7 |
| VEGF-A | 14.3 |
| VEGF-D | 14.6 |

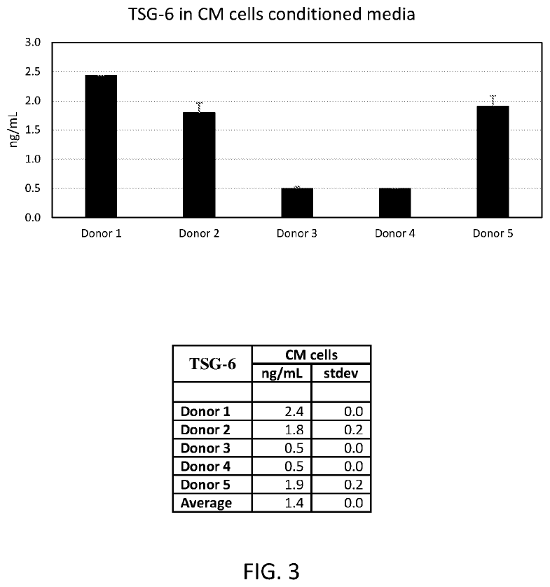

TSG-6 levels secreted by fresh minced chorion tissue and isolated from chorion cells. Isolated chorion cells were incubated overnight at 37C 5% CO₂ in DMEM supplemented with 10% Fetal Bovine Serum (FBS) 1% Penicillin Streptomycin. The presence of TSG-6, an anti-inflammatory protein, in conditioned media is determined by ELISA. (FIG. 3) In some aspects, the levels of TSG-6 can range from 0.1ng/ml to 10 ng/ml.

### C. Example 3

### A Composition Comprising a Minced Chorionic Matrix and a Devitalized Homogenized UC Matrix, Wherein the Non-Homogenized Chorionic Matrix Comprises Viable Cells, Wherein the Composition Does Not Comprise Trophoblasts or an Amniotic Matrix

1. Characterization of Minced Chorion and Umbilical Cord Matrix.

The presence of angiogenic and other growth factors in chorion cell, amnion and umbilical cord lysates was determined by multiplex assays (Luminex). Protein estimation was quantified by BCA assay and values were reported as picogram of target per milligram total protein (Table 3).

TABLE 3

| | CM/UC Matrix |
|---|---|
| Target | pg/mg |
| Angiogenin | 673.68 |
| BMP-7 | 222.22 |
| FGF-23 | 4.32 |
| PDGF-AA | 11.72 |
| TIMP-1 | 8896.05 |
| VEGF-C | 109.35 |
| BMP-4 | 13.25 |
| FGF basic | >368.44 |
| Osteopontin | 3623.36 |
| PDGF-BB | 37.05 |
| VEGF-A | 63.7 |
| IFN-gamma | 51.85 |
| IL-2 | 138 |
| IL-6 | 13619.94 |
| TNF-alpha | 8.73 |
| IL-1 beta | 316.81 |
| CCL2/MCP-1 | 55.55 |
| IL-1ra | 1355.77 |

2. Inhibition of TNF-α secretion by activated THP-1 cells.

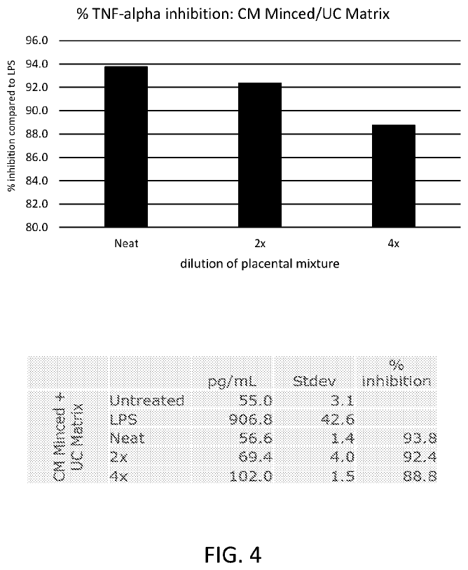

The anti-inflammatory capabilities of a composition comprising a minced chorionic matrix and a devitalized homogenized UC matrix, wherein the non-homogenized chorionic matrix comprises viable cells, wherein the composition does not comprise trophoblasts or an amniotic matrix were characterized by the inhibition of TNFα produced by Lipopolysaccharide (LPS) activated THP-1 cells. THP-1 cells were plated in a 24 well plate and stimulated with lug/mL of LPS in DMEM growth media and co-cultured neat or varying dilutions (2×, 4×) of minced chorion and umbilical cord matrix (CM+UC). Untreated THP-1 and LPS treated THP-1 cells represented positive and negative controls respectively. The culture was incubated overnight at 37° C. with 5% CO2. After overnight stimulation and treatment, the cell media was collected and the amount of TNFα produced by the THP-1 cells after LPS treatment is compared to the CM+UC treatments and reported as % inhibition vs the control (FIG. 4). TNFα is quantified by Luminex and/or ELISA (R&D Systems).

FIG. 4 is a titration experiment showing the inhibitory effect of CM minced/UC matrix even upon a 4-fold dilution. The inhibitory effect can be observed even when the samples are diluted to 10-fold. Similar to the experiment of FIG. 1, THP1 cells are representatives of the inflammatory cells that infiltrate into the site of injury. These cells produce inflammatory cytokines like TNFα when triggered by stimulus, i.e. LPS. LPS represents an infectious component, commonly seen in an injured tissue. In the experiment, the positive control, TNFα released by THP1 cells upon LPS treatment, is representative of the TNFα present in an inflammatory microenvironment. Upon addition of CM/AM/UC, the level of TNFα response is diminished and this is quantified as percent inhibition vs control. The objective is to decrease TNFα levels upon treatment.

### D. Example 4

### A Composition Comprising an Isolated Chorionic Cells (CM Cells) and a Devitalized Homogenized Umbilical Cord (UC) Matrix, Wherein the Composition Does Not Comprise Trophoblasts or an Amniotic Matrix

Characterization of chorion cells and umbilical cord matrix. The presence of angiogenic and other growth factors in chorion cells (CM cells) and umbilical cord (UC) lysates was determined by multiplex assays (Luminex) and reported as picogram of target per milligram total protein (Table 4).

TABLE 4

| | CM cells/UC |
|---|---|
| Target | pg/mg |
| IL-10 | 2.9 |
| FGF basic | 199.8 |
| Angiogenin | 2813.9 |
| Angiopoietin | 19.3 |
| HGF | 72.1 |
| PDGF-AA | 2.7 |
| PDGF-BB | 0.9 |
| VEGF-A | 6.8 |
| VEGF-D | 9.7 |

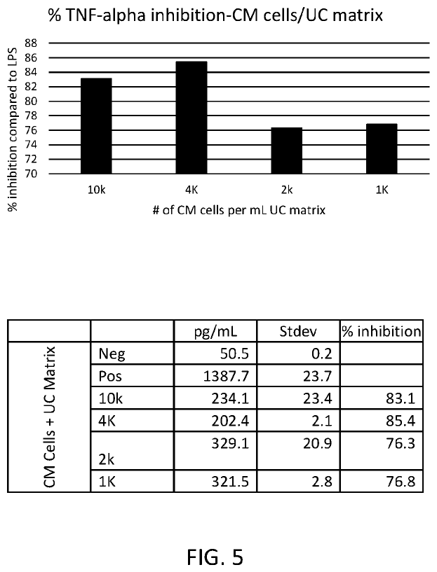

Inhibition of TNF-α secretion by activated THP-1 cells. The anti-inflammatory capabilities of a composition comprising an isolated chorionic cells and a devitalized homogenized UC matrix, wherein the composition does not comprise trophoblasts or an amniotic matrix were characterized by the inhibition of TNFα produced by Lipopolysaccharide (LPS) activated THP-1 cells. THP-1 cells were plated in a 24 well plate and stimulated with lug/mL of LPS in DMEM growth media and co-cultured with varying chorion cell densities (10k,4k, 2k, 1k) in umbilical cord matrix (CM cells +UC). Untreated THP-1 and LPS treated THP-1 cells represented positive and negative controls respectively. The culture was incubated overnight at 37° C. with 5% CO2. After overnight stimulation and treatment, the cell media was collected and the amount of TNFα produced by the THP-1 cells after LPS treatment is compared to the CM cells +UC treatments and reported as % inhibition vs the control (FIG. 5). TNFα is quantified by Luminex and/or ELISA (R&D Systems).

FIG. 5 is a titration experiment showing the inhibitory effect of the CM cells down to 1000 cells/ml. The inhibitory effect can be observed even when the number of CM cells is 100cells/ml UC matrix.

### E. Example 5

### HSA v No HSA Minced Chorion Viability

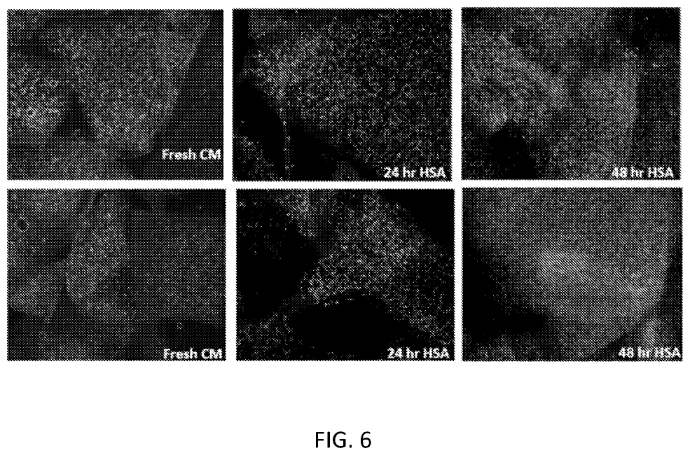

FIG. 6 shows the presence of live cells in fresh minced chorionic matrix (no Human Serum Albumin (HSA)), 24-hour and 48-hour HSA incubated minced chorion. Fresh CM incubated with HSA retains high viability even up to 78 hrs. In the absence of HSA, cell viability was heavily compromised (results not shown).

### F. Example 6

### Injectable Cellular Placental Formulation (ICPF) Works in Several Animal Models and Several Injuries

For this fibrosis model, ICPF was minced CM (cellular component) in AM+UC matrix. This formulation is also applicable to other formulations of AM/CM/UC combination.

1. Bleomycin Induced Skin Fibrosis Model in a C57B1/6 Mouse

A subcutaneous injection of bleomycin causes dermal fibrosis. A dermal injection with ICPF prior to bleomycin injury prevents the extent of dermal fibrosis. Histological analysis showed evident epidermal hyperplasia and myofiber degeneration in the control group and no dermal fibrosis in the ICPF-treated animals. Next Generation Sequencing (NGS) analysis of the miRNAs confirmed pathological changes in the ICPF-treated group. The threshold used to screen up- or downregulated miRNAs was log fold change≥1.5 and p value≤0.0001. The most differentially expressed known miRNAs were mmu-miR-376c-3p (up-regulated), mmu-miR-299a-5p (up-regulated), mmu-miR-184-3p (down-regulated), mmu-miR-147-3p (down-regulated), mmu-miR-3473e (down-regulated), and mmu-miR-146b-3p (down-regulated).

2. Hind Limb Ischemia Model in a Rat

A 2 cm segment of femoral artery and vein was removed from a rat to induce ischemia. ICPF was injected into the Gracilis muscle distal from vessel ligation. Doppler imaging was conducted up to 35 days post-surgery and confirmed there was increased hindlimb perfusion in ICPF group.

3. Fistula-in-ano Model in a Porcine

Three mature fistulas in-ano were created in-ano in pigs. Injection of either ICPF (particulate) or ICPF (minced), or saline were injected around the tract and pigs were observed over 2 weeks. Closure of fistula was observed in ICPF (minced and particulate) group but not in the saline group. The particulate formulation was isolated CM cells in AM/UC matrix. The minced formulation was minced CM in AM/UC matrix

4. Biodistribution Model in a Rat

Intramuscular injection of ICPF on the thigh muscle of the flank was performed. Next, on Days 3, 8, and 21, post injection, tissue from the injection site, liver, and lung was harvested. Detection of ICPF was not found at sites other than the site of delivery at Days 3, 8, and 21 post-injection. Upon histological evaluation of the harvested tissues, no ICPF presence was observed other than at the site of delivery. Histological evidence of ICPF at the site of delivery at Day 3, 8, and 21 post-delivery. ICPF was minced CM in AM/UC matrix.

5. Chemical Induced Liver Injury Model in a C57B1/6 Mouse

Thioacetamide was administered intraperitoneally 3 times a week for 6 weeks to mice. Tissues were collected for histology after 6 weeks of chemical treatment. ICPF was delivered 2 weeks prior to beginning of the thioacetamide treatment regimen. ICPF was evaluated for ability to prevent occurrence of fibrosis. Thioacetamide causes generation of reactive oxygen species and in combination with inflammation results in tissue fibrosis. ICPF is protective against ROS generation and inflammation. ICPF in this case was CM cells in AM/UC matrix. And the same can be extended to other iteration of the formulation of AM/UC/CM. Cells+matrix formulation has some degree of anti-fibrosis effect in vivo.

### G. Example 7

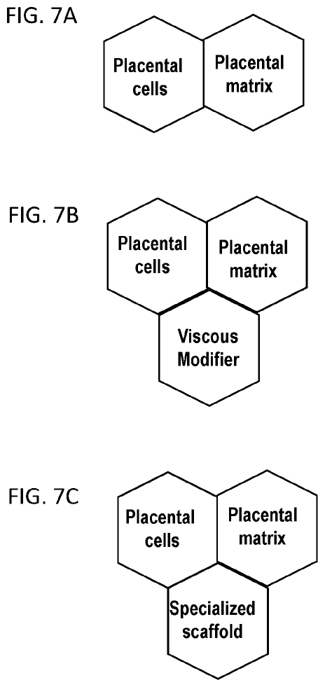

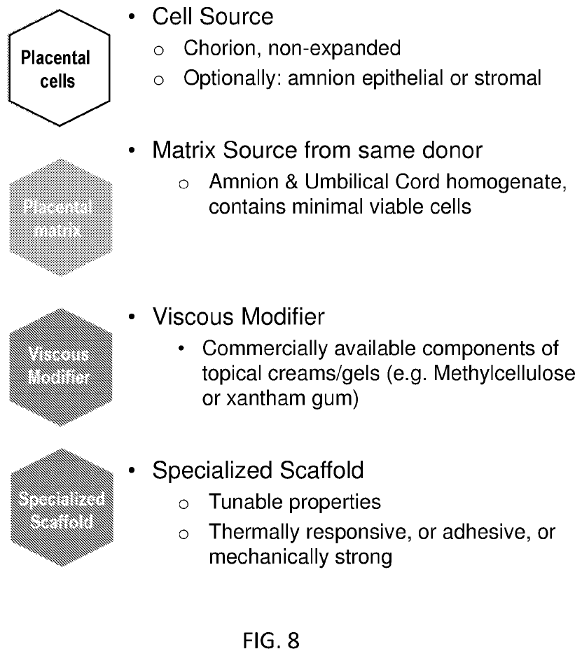

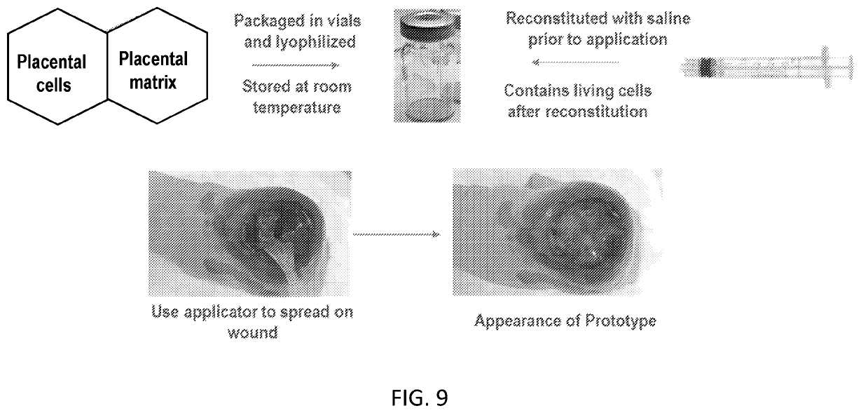

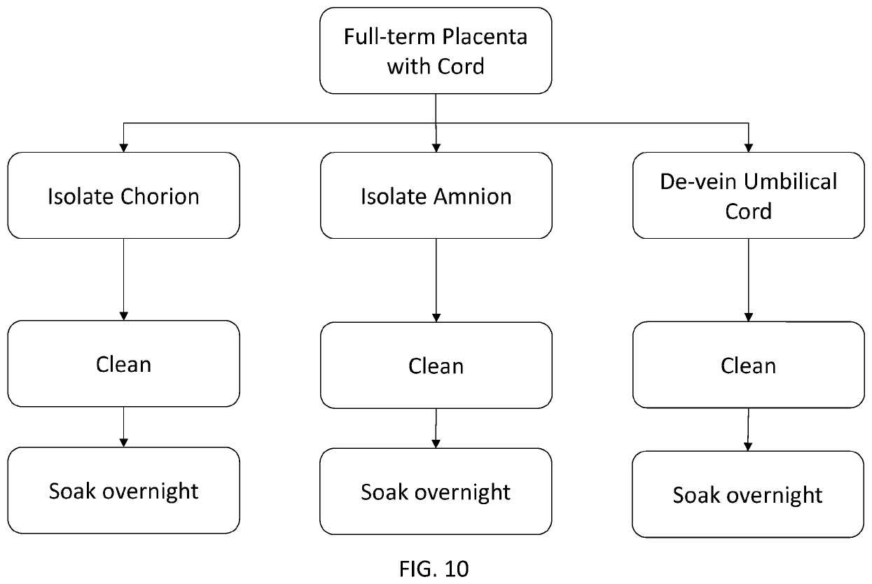

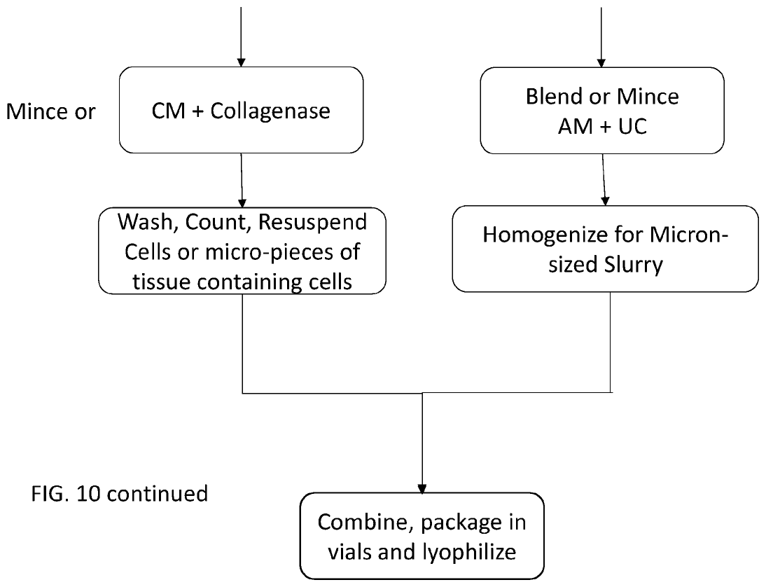

FIG. 7-FIG. 10 provide examples of the platform building blocks and how they form compositions. They also show an example of a method of processing a placenta and umbilical cord in order to produce an example of one of the disclosed compositions.

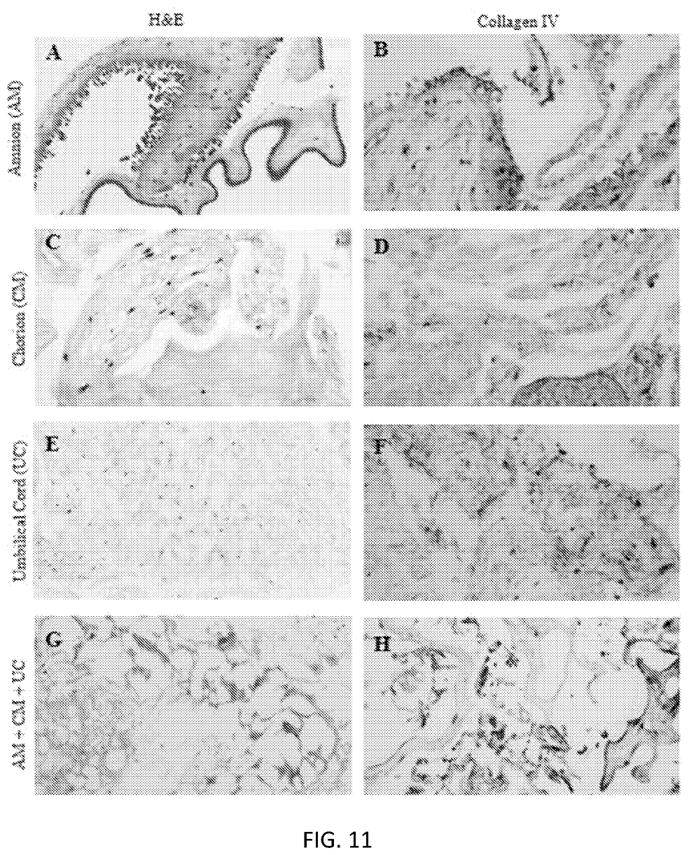

FIG. 11 shows the histological appearance of compositions. Representative pictures of H&E-stained sections of (A) Amnion (C) Chorion (E) Umbilical cord (G) the viable compositions (AM+CM+UC). Representative pictures of Collagen IV stained sections of (B) Amnion (D) Chorion (F) Umbilical cord (H) the viable compositions (AM+CM+UC). All images were taken at 20+ magnification.

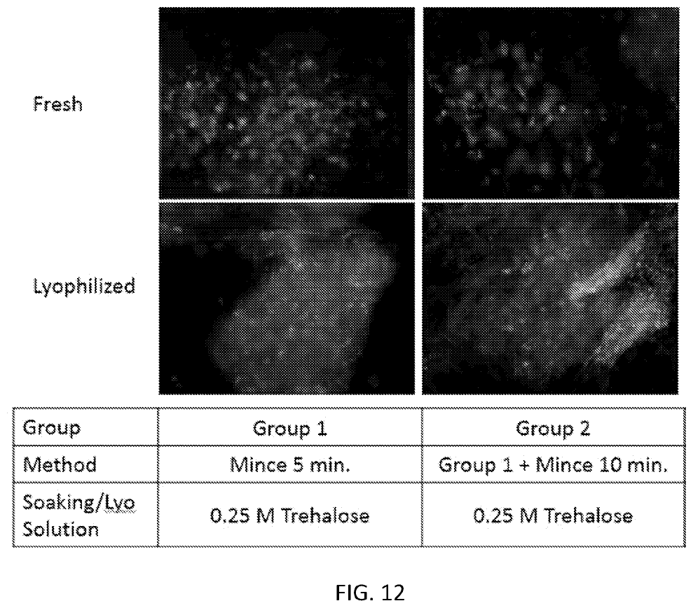

FIG. 12 shows cell viability of the non-homogenized chorionic component of the compositions after mincing, both fresh and after preservation by lyophilization. All samples were treated with the same solutions and lyophilized in the same manner. Each group was processed from the same starting material and represents samples taken in succession during a mincing process. Cell viability of the lyophilized group was nearly equivalent ˜90% of the starting material viability, and estimated to be 80-85% total cell viability for both groups.

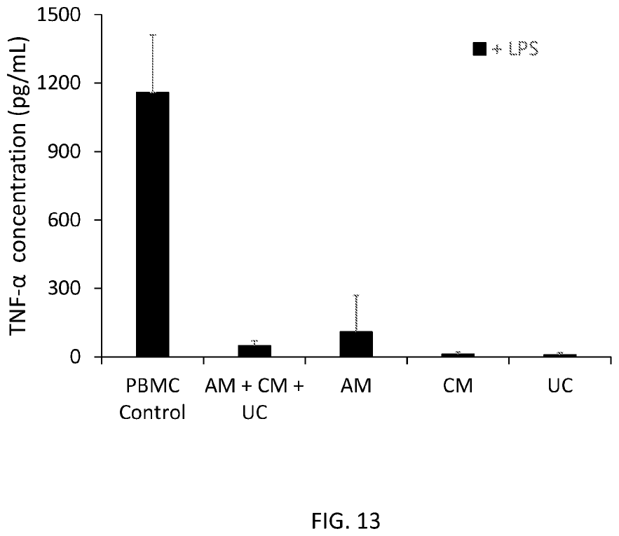

FIG. 13 demonstrates the lack of an immune response exhibited by the compositions, which is a result of the selective depleting or devitalizing of immunogenic cell types (lymphocytes, macrophages, endothelial cells, etc.) during the cryopreservation and/or lyophilization processes.

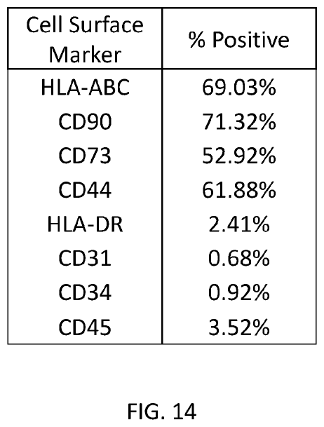

FIG. 14 is a table summarizing the FACS analysis of cells isolated from the non-homogenized viable chorionic component of the compositions for one lot. As shown, the cells are all negative (<5% positive) for markers of immunogenic cell types (CD45, CD31, HLA-DR) and the majority of cells (>50%) are also positive for cell surface markers typical of MSCs (CD90, CD73, CD44, HLA-ABC). This demonstrates the presence of viable chorionic stem cells in the compositions.

Those skilled in the art will recognize, or be able to ascertain using no more than routine experimentation, many equivalents to the specific embodiments of the method and compositions described herein. Such equivalents are intended to be encompassed by the following claims.

1. A composition comprising a minced chorionic matrix, a homogenized amniotic matrix, and a homogenized umbilical cord (UC) matrix, wherein the minced chorionic matrix comprises viable cells, wherein the composition does not comprise trophoblasts.

2. The composition of claim 1, wherein the homogenized amniotic matrix is devitalized.

3. The composition of any one of claims 1-2, wherein the homogenized umbilical cord (UC) matrix is devitalized.

4. The composition of any one of claims 1-3, wherein the chorionic matrix, homogenized amniotic matrix and homogenized UC matrix are from the same donor.

5. The composition of any one of claims 1-4, wherein at least two of the chorionic matrix, homogenized amniotic matrix and homogenized UC matrix are from the same donor.

6. A composition comprising isolated, viable chorionic cells, a homogenized amniotic matrix, and a homogenized UC matrix, wherein the composition does not comprise trophoblasts.

7. The composition of claim 6, wherein the homogenized amniotic matrix is devitalized.

8. The composition of any one of claims 6-7, wherein the homogenized UC matrix is devitalized.

9. The composition of any one of claims 6-8, wherein the homogenized amniotic matrix and homogenized UC matrix are derived from the same donor.

10. The composition of any one of claims 6-9, wherein the isolated chorionic cells and homogenized amniotic matrix are from the same donor.

11. The composition of any one of claims 6-9, wherein the isolated chorionic cells and homogenized UC matrix are from the same donor.

12. The composition of any one of claims 6-9, wherein the isolated chorionic cells, homogenized amniotic matrix, and homogenized UC matrix are from the same donor.

13. The composition of any one of claims 6-12, wherein the isolated chorionic cells comprise greater than or equal to 100,000 viable cells/ml.

14. The composition of any one of claims 6-13, wherein the isolated chorionic cells have not been culturally expanded.

15. The composition of any one of claims 6-14, further comprising a chorionic matrix.

16. The composition of claim 15, wherein the chorionic matrix is non-homogenized.

17. The composition of any one of claims 15-16, wherein the chorionic matrix is minced.

18. The composition of claim 17, wherein the minced chorionic matrix comprises native, viable cells.

19. The composition of any one of claims 17-18, wherein the minced chorionic matrix comprises viable cells that have not been culturally expanded.

20. A composition comprising a minced chorionic matrix and a homogenized UC matrix, wherein the minced chorionic matrix comprises viable cells, wherein the composition does not comprise trophoblasts or an amniotic matrix.

21. The composition of claim 20, wherein the homogenized UC matrix is devitalized.

22. The composition of any one of claims 20-21, wherein the minced chorionic matrix and homogenized UC matrix are from the same donor.

23. The composition of any one of claims 20-22, wherein the minced chorionic matrix is non-homogenized.

24. A composition comprising isolated, viable chorionic cells, and a homogenized UC matrix, wherein the composition does not comprise trophoblasts or an amniotic matrix.

25. The composition of claim 24, wherein the homogenized UC matrix is devitalized.

26. The composition of any one of claims 24-25, wherein the isolated chorionic cells and homogenized UC matrix are from the same donor.

27. The composition of any one of claims 24-26, wherein the isolated chorionic cells comprise greater than or equal to 100,000 viable cells/ml.

28. The composition of any one of claims 24-27, wherein the isolated chorionic cells have not been culturally expanded.

29. The composition of any one of claims 24-28, further comprising a chorionic matrix.

30. The composition of claim 29, wherein the chorionic matrix is non-homogenized.

31. The composition of any one of claims 29-30, wherein the chorionic matrix is minced.

32. A composition comprising isolated, viable chorionic cells, a homogenized UC matrix, and a homogenized amniotic matrix wherein the composition does not comprise trophoblasts.

33. The composition of claim 32, wherein the homogenized UC matrix is devitalized.

34. The composition of any one of claims 32-33, wherein the isolated chorionic cells and homogenized UC matrix are from the same donor.

35. The composition of any one of claims 32-34, wherein the isolated chorionic cells comprise greater than or equal to 100,000 viable cells/ml.

36. The composition of any one of claims 32-35, wherein the isolated chorionic cells have not been culturally expanded.

37. The composition of any one of claims 32-36, further comprising a chorionic matrix.

38. The composition of claim 37, wherein the chorionic matrix is non-homogenized.

39. The composition of any one of claims 37-38, wherein the chorionic matrix is minced.

40. The composition of any one of claims 1-5, 20-23, 29-31, and 37-39, wherein the chorionic matrix comprises viable cells.

41. The composition of claim 40, wherein the chorionic matrix comprises native, viable cells.

42. The composition of any one of claims 40-41, wherein the chorionic matrix comprises viable cells that have not been culturally expanded.

43. The composition of any one of claims 1-5, 15-23, 29-31, and 37-42, wherein the chorionic matrix comprises greater than or equal to 100,000 viable cells/ml.

44. The composition of any one of claims 1-43, wherein the composition comprises viable chorionic stem cells, amniotic stem cells, fibroblasts, epithelial cells, or a combination thereof.

45. The composition of any one of claims 1-44, wherein the composition is formulated as a cream, gel, oil, ointment, or lotion.

46. The composition of any one of claims 1-45, further comprising a viscous modifier.

47. The composition of claim 46, wherein the viscous modifier is hyaluronic acid, methylcellulose, carboxymethylcellulose, xanthum gum, pluronics, thermally responsive polymers and proteins, fibronectins, laminins, collagens, chitosan, or chondroitin sulfate.

48. The composition of any one of claims 1-47 further comprising viable, isolated amniotic cells.

49. The composition of any one of claims 1-48, further comprising a scaffold.

50. The composition of claim 49, wherein the scaffold is natural or synthetic.

51. The composition of any one of claims 49-50, wherein the scaffold is derived from skin, hyaline cartilage, meniscus, intervertebral discF, or bone.

52. The composition of any one of claims 49-51, wherein the scaffold is a natural or synthetic polymer.

53. The composition of any one of claims 1-52, wherein the homogenized UC matrix comprises de-veined UC tissue.

54. The composition of any one of claims 1-53, wherein the composition is cryopreserved.

55. The composition of any one of claims 1-54, wherein the composition comprises a cryopreservation solution.

56. The composition of any one of claims 1-55, wherein the composition is lyophilized.

57. The composition of any one of claims 1-56, wherein the composition further comprises a pharmaceutically acceptable excipient.

58. A pharmaceutical composition comprising any one of the compositions of claims 1-57 and a pharmaceutically acceptable carrier.

59. A method of making the composition of any one of claims 1-5, 20-22, 35-57 comprising:
a) preparing a minced chorionic matrix;
b) preparing a homogenized UC matrix; and
c) combining the minced chorionic matrix and the homogenized UC matrix.

60. The method of claim 59 further comprising preparing a homogenized amniotic matrix and combining the homogenized amniotic matrix with the minced chorionic matrix and the homogenized UC matrix.

61. A method of making the composition of any one of claims 6-19, 23-33 comprising:
a) preparing isolated chorionic cells;
b) preparing a homogenized UC matrix; and
c) combining the isolated chorionic cells and the homogenized UC matrix.

62. The method of claim 61, wherein preparing isolated chorionic cells comprises isolating chorionic cells from chorionic tissue.

63. The method of any one of claims 61-62 further comprising the step of preparing a homogenized amniotic matrix and combining with the isolated chorionic cells and the homogenized UC matrix.

64. The method of any one of claims 61-63, further comprising preparing a non-homogenized chorionic matrix.

65. The method of claim 64, wherein preparing a non-homogenized chorionic matrix comprises mincing chorionic tissue.

66. The method of any one of claims 64-65, wherein preparing a non-homogenized chorionic matrix comprises removing a trophoblast layer.

67. The method of any one of claims 61-66, wherein the isolated chorionic cells and UC tissue are derived from the same donor.

68. The method of any one of claims 59-67, further comprising adding a viscous modifier.

69. The method of any one of claims 59-68, wherein the homogenized UC matrix comprises de-veined UC tissue.

70. The method of any one of claims claim 59-69, further comprising adding a scaffold.

71. The method of claim 70, wherein the scaffold is natural or synthetic.

72. The method of any one of claims 70-71, wherein the scaffold is derived from a meniscus, a disc, or bone.

73. The method of claim 71, wherein the scaffold is a natural or synthetic polymer.

74. The method of any one of claims 60, 63-73 further comprising prior to preparing a homogenized amniotic matrix, isolating epithelial cells from the amniotic matrix.

75. The method of claim 74 further comprising combining the isolated amniotic epithelial cells to the combined isolated chorionic cells, the homogenized amniotic matrix, and the homogenized UC matrix.

76. The method of any one of claims 59-75, wherein the method further comprises lyophilizing the combined isolated chorionic cells and UC matrix.

77. A method of treating a tissue injury or chronic pain comprising administering the composition of one of claims 1-58 to an area of a subject comprising a tissue injury.

78. The method of claim 77, wherein the tissue injury is osteoarthritis, cartilage repair, meniscus repair, intervertebral disc repair, plantar fasciitis, carpal tunnel, tendonitis, synovitis, ruptured or torn Achilles tendon, critical limb ischemia, ulcers, pyoderma gangrenosum, epidermolysis bullosa, surgical adhesions, plastic surgery or other wounds.

79. The method of any one of claims 77-78, wherein the composition is administered by injecting the composition to the area of a subject comprising a tissue injury or local region of pain or inflammation.

80. The method of any one of claims 77-79, wherein the composition is administered by applying the composition topically to an area of a subject comprising the tissue injury or pain or inflammation.

81. The method of any one of claims 77-80, wherein the composition is administered by implanting the composition to the area of a subject comprising a tissue injury.
